(19) United States
(12) Patent Application Publication
(10) Pub. No.: US 2023/0165972 A1
(43) Pub. Date: Jun. 1, 2023

(54) CANCER THERAPY WITH MICROBUBBLES

(71) Applicant: SONOTARG LIMITED, Belfast (GB)

(72) Inventors: John CALLAN, Ballycastle Antrim (GB); Anthony MCHALE, Ballycastle Antrim (GB)

(73) Assignee: SONOTARG LIMITED, Belfast (GB)

(21) Appl. No.: 17/915,251

(22) PCT Filed: Mar. 30, 2021
(86) PCT No.: PCT/GB2021/050787

§ 371 Date: Sep. 28, 2022

(30) Foreign Application Priority Data
Mar. 30, 2020 (GB) 2004629.8

(51) Int. Cl. A61K 47/69; A61K 41/00; A61K 47/64; A61P 35/00; A61P 1/18

(52) U.S. Cl. CPC A61K 47/6925; A61K 41/0047; A61K 47/64; A61P 1/18; A61P 35/00

(57) ABSTRACT

The invention relates to a microbubble-chemotherapeutic agent complex comprising a microbubble carrying a combination of chemotherapeutic agents for use in a method of treating cancer in a patient, wherein said combination of chemotherapeutic agents comprises: (a) a 5-fluoropyrimidine or a derivative thereof; (b) irinotecan or a derivative thereof; and (c) a platinum-based chemotherapeutic agent or a derivative thereof; and wherein said method comprises simultaneous, separate or sequential administration of folinic acid or a derivative thereof. The invention is particularly suitable for use in the treatment of deep-sited tumours and associated metastatic disease, for example in the treatment of pancreatic cancer. The invention further relates to the microbubble- chemotherapeutic agent complexes themselves, to methods for their preparation and to pharmaceutical compositions which contain them, optionally in combination with folinic acid or a folinic acid derivative.

### TECHNICAL FIELD

The present invention relates generally to the treatment of cancer and, more specifically, to the treatment of deep-sited tumours and associated metastatic disease which remain difficult to treat due to the extreme toxicity of conventional multi-drug based chemotherapies. In particular, it relates to the treatment of pancreatic cancer, for example pancreatic adenocarcinoma.

More particularly, the invention relates to a method for the treatment of cancer in which a combination of highly toxic chemotherapeutics is loaded onto a microbubble and delivered by rupturing the bubble at the target site using low intensity ultrasound. In this method, any non-cytotoxic therapeutics are co-administered. Since these are non-toxic, it is not necessary for these to be delivered on a microbubble.

The invention further relates to drug-loaded microbubbles, i.e. to microbubbles which carry the combination of highly toxic chemotherapeutics, to methods for their preparation, and their use in methods of medical treatment.

### BACKGROUND OF THE INVENTION

Conventional treatment of deep-sited tumours typically involves surgery, chemotherapy, radiotherapy or combinations of all of these. All of these can result in various complications. The development of more targeted and less invasive therapies with improved efficacy remains highly sought after.

Pancreatic cancer is one example of a deep-sited tumour and is one of the most lethal types of cancer. It accounts for about 2% of all cancers with a five year survival rate of 15-21% in patients who have a surgical resection followed by systemic chemotherapy. Pancreatic adenocarcinoma (PAC) accounts for about 85% of pancreatic cancer cases.

Surgery is the only potentially curative option for patients with pancreatic cancer, but is only possible in 15-20% of patients since non-specific symptoms and the aggressive nature of the disease generally lead to late diagnosis. Most patients have locally advanced or metastatic pancreatic adenocarcinoma (mPAC) at diagnosis, and 60-90% of patients who have undergone resection will develop locally recurrent or metastatic disease despite surgery and adjuvant treatment.

The most common first-line treatments for pancreatic adenocarcinoma are gemcitabine in combination with albumin-bound paclitaxel (nab-paclitaxel), gemcitabine monotherapy, and infusional FOLFIRINOX. FOLFIRINOX is a chemotherapy regimen involving systemic administration of folinic acid / leucovorin (FOL), 5-fluorouracil or “5-FU” (F), irinotecan (IRIN), and oxaliplatin (OX). Folinic acid is a vitamin B derivative which is used as a 5-FU adjuvant. It enhances the effect of 5-FU by inhibiting thymidylate synthase. 5-FU is a pyrimidine analogue and anti-metabolite which incorporates into the DNA molecule and stops DNA synthesis. Irinotecan is a topoisomerase inhibitor which prevents DNA from uncoiling and duplicating, and oxaliplatin is a platinum-based chemotherapeutic agent which inhibits DNA repair and/or DNA synthesis.

FOLFIRINOX treatment is recommended for patients with advanced pancreatic cancer and provides improved survival rates for those who have the disease (Lee et al., Chemotherapy 59: 273-9, 2013). However, FOLFIRINOX is a highly toxic combination of drugs with extreme off-target toxicity and these complications strongly influence decisions in the treatment of older patients (65 years and older) and/or those having a poor physical performance status. Guidelines issues by The European Society for Medical Oncology (ESMO) only recommend its use for those patients who are otherwise fit and healthy, i.e. who have a good Eastern Cooperative Oncology Group performance status (ECOG PS). Adverse events associated with the treatment include gastrointestinal disorders, myocardial infarction, angina, neutropenia, anaemia, thrombocytopenia, hyperbilirubinemia, hepatic or renal dysfunction, and weight loss. This leads to additional costs relating to toxicity management.

First-line FOLFIRINOX and gemcitabine-based treatments can extend survival by several months in patients with pancreatic adenocarcinoma. However, survival rates remain low and there is an ongoing need for alternative treatment methods which are minimally invasive and which can improve survival whilst minimising adverse events. Such methods would have obvious socio-economic benefits, e.g. in terms of reduced patient trauma and reduced treatment expense, including reduced costs associated with any hospital stay. Any treatment method having reduced toxicity can also be used to treat a wider patient group, i.e. not just those patients having a good ECOG performance status.

### SUMMARY OF THE INVENTION

The inventors now propose ultrasound-targeted-microbubble-destruction (“UTMD”) to deliver conventional FOLFIRINOX therapy. This involves the use of a microbubble to carry the toxic chemotherapeutics (FIRINOX) together with separate administration of folinic acid / leucovorin (FOL). Folinic acid is not a chemotherapeutic, but is used to enhance 5-FU activity. It is non-toxic and is well tolerated so need not be carried on the microbubble.

UTMD is an emerging field in drug delivery and involves the use of low intensity ultrasound to rupture microbubbles at a target site, releasing the attached payload and encapsulated gas in a localised manner. An additional benefit is the motion of the microbubbles in the ultrasound field, which enhances microscale mass transport through impermeable tissue thus assisting payload delivery across the tumour stroma. When used to deliver chemotherapeutics, such targeted chemotherapy also reduces the exposure of normal tissues to the highly cytotoxic drugs.

Based on data obtained from studies in mice and reported herein, the inventors have now unexpectedly found that the use of UTMD to deliver the FIRINOX chemotherapeutic agents provides further advantages in the treatment of pancreatic cancer compared to the conventional “standard-of-care” chemotherapy. Specifically, they have found that this improves tumour growth delay when used at doses that are significantly lower than those used in the standard treatment. That this improvement is seen at doses which, when used in the standard treatment, would be ineffective (i.e. at “sub-therapeutic” doses) is surprising. The ability to employ lower (e.g. sub-therapeutic) doses of the chemotherapeutic agents significantly reduces the toxicity of the treatment. This provides a significant advance in the use of the FOLFIRINOX treatment for pancreatic cancer. For example, it potentially allows further rounds of FOLFIRINOX treatment for patients who are eligible for the treatment under the current guidelines. It also has the potential for the treatment to be extended to patients who would otherwise not be considered eligible, for example patients who do not have a good ECOG performance status. The inventors’ findings are also expected to provide significant benefits in terms of improved survival rates for patients with pancreatic cancer and a better quality of life during treatment due to the significant reduction in side effects.

As reported herein, these initial findings have also been verified in the treatment of colon cancer in a mouse model of the disease and can thus be expected to extend to other cancers. Thus, whilst the present disclosure is primarily focused on the treatment of pancreatic cancer, this is not intended to be limiting. Any of the methods, uses, compositions, products and kits herein described are considered to be suitable for the treatment of other cancers and associated metastatic disease, in particular for the treatment of other solid tumours.

### DETAILED DESCRIPTION OF THE INVENTION

In one aspect, the invention provides microbubbles carrying a combination of chemotherapeutic agents for use in a method of treating cancer, wherein said combination of chemotherapeutic agents comprises:
(a) a 5-fluoropyrimidine or a derivative thereof;
(b) irinotecan or a derivative thereof; and
(c) a platinum-based chemotherapeutic agent or a derivative thereof; and wherein said method comprises simultaneous, separate or sequential use of folinic acid or a derivative thereof.

The method of treatment herein described involves administration of a plurality of microbubbles each carrying the defined combination of chemotherapeutics, together with the co-administration of folinic acid or a derivative thereof. The defined chemotherapeutic agents are carried by the bubble as herein described, for example by encapsulation and/or attachment to the bubble. The drug-loaded microbubbles are referred to herein as “microbubble-chemotherapeutic agent complexes” or, simply, “microbubble-complexes”. Ultrasound is used to rupture the microbubbles so that they release their shell fragments and core gas at the site of destruction. In this way, the delivery of the chemotherapeutics is mainly confined to the target site by exposure of that site to an appropriate ultrasound stimulus. In addition to the localised deposition of the drug payloads, the physical forces that accompany microbubble inertial cavitation (i.e. micro-streaming and micro-jetting) enhances dispersion of the shell fragments into the target tissue.

As used herein, the term “microbubble” is intended to refer to a microsphere comprising a shell having an approximately spherical shape and which surrounds an internal void which comprises a gas or mixture of gases. The “shell” refers to the membrane which surrounds the internal void of the microbubble. It is intended that the microbubble will be ultrasound-responsive, i.e. it can be ruptured by application of ultrasound thereby releasing its payload at the desired target site.

As used herein, the terms “chemotherapeutic agent” and “chemotherapeutic” are used interchangeably and are intended to refer to any compound useful in the treatment of cancer.

As used herein, the term “derivative” includes any chemically modified form of an active compound, e.g. a chemotherapeutic agent. Specifically, it includes any prodrug form of the compound which is converted metabolically to the compound and is thus essentially equivalent thereto. The term “derivative” also includes pharmaceutically acceptable salts of any of the active agents herein described. As used herein, the term “prodrug” refers to a derivative of an active compound which undergoes a transformation under the conditions of use, for example within the body, to release an active drug. A prodrug may, but need not necessarily, be pharmacologically inactive until converted into the active drug. The term “prodrug” extends to any compound which, under physiological conditions, is converted into any of the active compounds herein described. Suitable prodrugs include compounds which are hydrolysed under physiological conditions to the desired molecule, or which are transformed to the active drug by the action of enzymes in vivo.

The term “pharmaceutically acceptable salt” as used herein refers to any pharmaceutically acceptable organic or inorganic salt of any of the compounds herein described. Examples of suitable pharmaceutically acceptable salts are well known to those of skill in the art.

5-fluoropyrimidine chemotherapeutics are well known in the art and any of these may be used in the invention, or any chemically modified form thereof which retains the desired anti-cancer activity. 5-fluorouracil (5-FU) and its prodrug forms are particularly suitable for use in the invention, for example 5-fluorouridine (5-FUR), capecitabine, carmofur, doxifluridine, and tegafur. Any of these may be used in the form of a derivative as herein described, e.g. as a pharmaceutically acceptable salt.

Irinotecan is 7-ethyl-10-[4-(1-piperidino)-1-piperidino]carbonyloxycampothecin. It has the following structure:

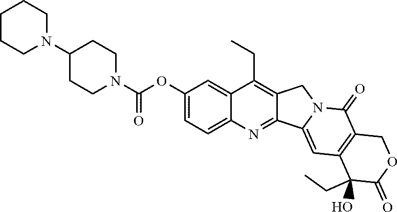 [SMILES: CCc1c2c(nc3ccc(OC(=O)N4CCC(N5CCCCC5)CC4)cc13)-c1cc3c(c(=O)n1C2)COC(=O)[C@]3(O)CC]

For use in the invention, irinotecan may be used in the form of its free base. Alternatively, it may be used in the form of a pharmaceutically acceptable salt such as, but not limited to, its hydrochloride salt.

Platinum-based chemotherapeutics (also known as “platins”) are well known in the art and any of these may be used in the invention, or any chemically modified form thereof which retains the desired anti-cancer activity. These chemotherapeutic agents include both Pt(ll) and Pt(IV) complexes. Non-limiting examples of platins suitable for use in the invention include cisplatin, oxaliplatin, carboplatin, satraplatin, picoplatin, tetraplatin, platinum-DACH (DACH = 1,2-diaminocyclohexane) and derivatives thereof (for example, chemically modified forms, and pharmaceutically acceptable salts). In one embodiment, oxaliplatin or a derivative thereof may be used. One example of a derivative of oxaliplatin which may be used in the invention is its corresponding diol, i.e. Pt(DACH)(Ox)(OH)₂ (DACH = 1,2-diaminocyclohexane; Ox = oxalate), which features the bidentate DACH ligand, a bidentate oxalate group and two hydroxyl groups linked to Pt(IV):

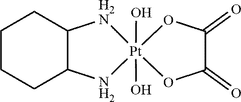

Folinic acid, also known as leucovorin, has the following structure:

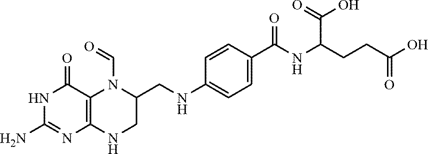 [SMILES: Nc1nc2c(c(=O)[nH]1)N(C=O)C(CNc1ccc(C(=O)NC(CCC(=O)O)C(=O)O)cc1)CN2]

For use in the invention, folinic acid can be used in the form of a pharmaceutically acceptable salt.

Microbubbles and methods for their preparation are well-known in the art. Examples of procedures for the preparation of microbubbles are described in, for example, Christiansen et al., Ultrasound Med. Biol., 29: 1759-1767, 2003; Farook et al., J. R. Soc. Interface, 6: 271-277, 2009; and Stride & Edirisinghe, Med. Biol. Eng. Comput., 47: 883-892, 2009, the contents of which are hereby incorporated by reference. Microbubbles comprise a shell which surrounds an internal void comprising a gas. Generally, these are approximately spherical in shape, although the shape of the microbubble is not essential in carrying out the invention and is therefore not to be considered limiting. As will be understood, the size of the microbubble should be such as to permit its passage through systemic circulation (e.g. the pulmonary system) following administration, e.g. by intravenous injection.

Microbubbles for use in the invention may typically have a diameter of less than about 200 µm, preferably in the range from about 0.1 to about 100 µm, e.g. from about 0.5 to about 100 µm. Particularly suitable for use in the invention are microbubbles having a diameter of less than about 10 µm, more preferably 1 to 8 µm, particularly preferably up to 5 µm, e.g. 1 to 3 µm or about 2 µm. The shell of the microbubble will vary in thickness depending on the materials used in its preparation and will typically range from about 5 to about 200 nm, e.g. from about 10 to about 200 nm. However, the precise thickness is not essential provided that the shell performs the desired function of retaining the gas core.

Materials which may be used to form the microbubbles should be biocompatible and suitable materials are well known in the art. Typically, the shell of the microbubble will comprise a surfactant, a polymer or a protein. Surfactants which may be used include any material which is capable of forming and maintaining a microbubble by forming a layer at the interface between the gas within the core and an external medium, e.g. an aqueous solution which contains the microbubble. A surfactant or combination of surfactants may be used. Those which are particularly suitable include lipids, in particular phospholipids. Polymer materials which are suitable for use in forming the shell of the microbubble include biocompatible polymers such as, but not limited to, poly(vinyl alcohol) (PVA), poly(D,L-lactide-co-glycolide) (PLGA), cyanoacrylate, poloxamers (Pluronics), chitosan and chitosan derivatives, or combinations thereof. Suitable proteins include albumin, particularly human serum albumin.

Suitable lipids may be of natural, semi-synthetic or synthetic origin. As will be understood, the lipids should be biocompatible. Lipids which are suitable for the preparation of a microbubble are known in the art and include any of those described herein. To produce a microbubble, the lipids may be amphiphilic in character, i.e. having both hydrophilic and hydrophobic properties. Suitable lipids include, but are not limited to, phospholipids, fatty acids, triglycerides, diglycerides, monoglycerides, sterols and sterol derivatives (e.g. cholesterol), sphingolipids (e.g. sphingomyelin), and combinations thereof. Lipids containing saturated and/or unsaturated fatty acid groups may be used. Long chain lipids are generally preferred, for example those containing fatty acid chains having from 10 to 30 carbon atoms, preferably 10 to 25 carbon atoms, e.g. from 12 to 22 carbons in either linear or branched form (preferably in linear form). Any fatty acids herein described may be fluorinated, i.e. these may include one or more (e.g. one) fluorine atoms. Examples of saturated fatty acids that may be present in the lipids include, for example, lauric, myristic, palmitic, stearic, and docosanoic (behenic) acids. Examples of unsaturated fatty acids that may be present include, for example, lauroleic, myristoleic, palmitoleic and oleic acids. Examples of branched fatty acids include, for example, isolauric, isomyristic, isoplamitic and isostearic acids. Saturated fatty acids, especially long chain fatty acids are generally preferred.

In one embodiment, phospholipids may be used to form the microbubbles. These consist of two hydrophobic fatty acid tail groups and a hydrophilic head containing a phosphate group. The head and tail groups are linked together by a glycerol backbone. The hydrophilic head consists of a phosphate group which may be modified with various organic molecules such as, for example, choline, ethanolamine or serine. The nature of the phospholipid is not particularly limited and includes phospholipids having both saturated and unsaturated fatty acid groups (including fatty acid groups which may be fluorinated). Saturated and unsaturated (including mono- and polyunsaturated) fatty acids include, but are not limited to, molecules having 10 to 30 carbon atoms, preferably 10 to 25 carbon atoms, e.g. 12 to 22 carbon. atoms, in either linear or branched form. Examples of fatty acids include any of those listed herein and any of these may optionally be fluorinated. Non-limiting examples of phospholipids suitable for use in the invention include the following and any mixtures thereof: phosphatidylcholines, e.g. dimyristoylphosphatidylcholine, dipalmitoylphosphatidylcholine, distearoylphosphatidylcholine, dioleoylphosphatidylcholine, dipentadecanoylphosphatidylcholine, dilauroylphosphatidylcholine, and 1,2-dibehenoyl-sn-glycero-3-phosphocholine (DBPC); phosphatidic acids; phosphatidylethanolamines, e.g. dioleoylphosphatidylethanolamine, and 1,2-distearoyl-sn-glycero-3-phosphoethanolamine (DSPE); phosphatidylserines; phosphatidylglycerols, e.g. diphosphatidylglycerols such as cardiolipin; and phosphatidylinositols.

Suitable phospholipids may be exemplified by the following compounds of formula (I), and their pharmaceutically acceptable salts:

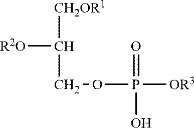

wherein:
R¹ and R², which may be the same or different, are saturated, mono- or polyunsaturated C₁₀₋₃₀ acyl groups, for example —CO—C₁₀₋₂₅ alkyl or —CO—C₁₀₋₂₅ alkenyl groups; and
R³ is selected from the following groups:

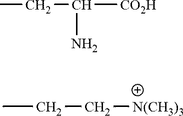

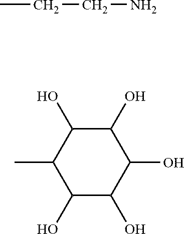

In formula (I), R¹ and R² will typically be derived from saturated fatty acids and are thus —CO—alkyl groups. These may be the same or different, e.g. selected from lauroyl, myristoyl, palmitoyl, stearoyl, oleoyl, linoleoyl, and behenoyl groups. In one embodiment, the lipids for use in the invention are selected from 1,2-dibehenoyl-sn-glycero-3-phosphocholine (DBPC), 1,2-distearoyl-sn-glycero-3-phosphoethanolamine (DSPE), and combinations thereof. In one embodiment a combination of lipids may be used to prepare the microbubble-complexes in which DBPC is present in an amount of at least 70, preferably at least 80, more preferably at 90 mol.% (based on the total amount of lipid).

The microbubble shell may further comprise components which aid in its attachment to one or more of the selected chemotherapeutic agents, optionally via a linking group or groups as herein described. In one embodiment, for example, the microbubble shell may be covalently coupled to biotin or a biotin residue via a molecular spacer, such as polyethylene glycol (PEG) (e.g. PEG-2000). This enables functionalisation of the surface of the microbubble with avidin which may then be conjugated to a biotinylated chemotherapeutic agent, to a biotinylated linking group which carries a chemotherapeutic agent, or to a biotinylated liposome which encapsulates a chemotherapeutic agent. Incorporation of a lipid-spacer-biotin conjugate (e.g. a lipid-PEG-biotin conjugate) in the shell of the microbubble may be achieved by appropriate functionalisation of one or more lipids prior to formation of the microbubble as herein described.

The microbubble shells may comprise single or multiple layers of the same or different materials. Multiple layers may, for example, be formed in cases where the basic shell material (e.g. a lipid) bears one or more polymers or polysaccharides, for example polyethylene glycol (PEG) or polyvinylpyrrolidone.

The microbubble shells may comprise further components which aid in accumulation of the microbubbles at the target site. For example, these may be functionalised such that these incorporate or have bound thereto a ligand or targeting agent which is able to bind to a target cell or tissue. Examples of suitable targeting agents include antibodies and antibody fragments, cell adhesion molecules and their receptors, cytokines, growth factors and receptor ligands. Such agents can be attached to the microbubbles using methods known in the art, e.g. by covalent coupling, the use of molecular spacers (e.g. PEG) and/or the avidin-biotin complex method. For example, the incorporation of a lipid-PEG-biotin conjugate in lipid-based microbubbles followed by the addition of avidin enables functionalisation of the microbubble surface with a biotinylated targeting ligand. However, the use of molecular targeting agents is not essential in order to achieve the desired targeting. In one embodiment, therefore, the microbubbles for use in the invention do not carry any ligand or targeting agent capable of binding to a target cell or tissue.

The gas provided within the core of the microbubble should be biocompatible. The term “gas” encompasses not only those substances which are gaseous at ambient temperature and pressure, but also those which are in liquid form under these conditions. Where the “gas” is liquid at ambient temperature this will generally undergo a phase change to a gas or vapour at a temperature of 38° C. or above. For any gas which is a liquid at ambient temperature, it is generally preferred that this will undergo a phase change to a gas at a temperature between about 38 and 45° C., preferably slightly above body temperature. For example, it may undergo a phase change when subjected to a stimulus, such as ultrasound, which causes a local increase in temperature. Any reference herein to “gas” should thus be considered to encompass not only gases and liquids, but also liquid vapours and any combination thereof, e.g. a mixture of a liquid vapour in a gas.

Gases which are suitable for incorporation within the microbubbles for use according to the invention include air, nitrogen, oxygen, carbon dioxide, hydrogen; inert gases such as helium, argon, xenon or krypton; sulphur fluorides such as sulphur hexafluoride, disulphur decafluoride; low molecular weight hydrocarbons such as alkanes (e.g. methane, ethane, propane, butane), cycloalkanes (e.g. cyclopropane, cyclobutane, cyclopentane), alkenes (e.g. ethylene, propene); and alkynes (e.g. acetylene or propyne); ethers; esters; halogenated low molecular weight hydrocarbons; and mixtures thereof. Examples of suitable halogenated hydrocarbons are those which contain one or more fluorine atoms and include, for example, bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane and perfluorocarbons. Examples of suitable fluorocarbon compounds include perfluorocarbons. Perfluorocarbons include perfluoroalkanes such as perfluoromethane, perfluoroethane, perfluoropropanes, perfluorobutanes, perfluoropentanes, perfluorohexanes and perfluoroheptanes; perfluoroalkenes such as perfluoropropene, perfluorobutenes; and perfluorocycloalkanes such as perfluorocyclobutane. Microbubbles containing perfluorinated gases, in particular, perfluorocarbons such as perfluoropropanes, perfluorobutanes, perfluoropentanes and perfluorohexanes are suitable for use in the invention due to their stability in the bloodstream. In one embodiment, the microbubbles for use in the invention may carry oxygen (e.g. oxygen gas). Since many cancers possess a pronounced hypoxic tumour environment which can also suppress the immune system, the use of oxygen-loaded microbubbles which simultaneously deliver oxygen and their attached payloads to tumours is particularly beneficial both from a therapeutic and immunological perspective.

Various methods for the formation of microbubbles are known in the art. Such methods include the formation of a suspension of the gas in an aqueous medium in the presence of the selected shell material. Techniques used to form the microbubble include sonication, high speed mixing (mechanical agitation), coaxial electrohydrodynamic atomisation and microfluidic processing using a T-junction (see e.g. Stride & Edirisinghe, Med. Biol. Eng. Comput., 47: 883-892, 2009). Sonication is widely used and generally preferred. This technique may be carried out using an ultrasound transmitting probe. More particularly, an aqueous suspension of the microbubble shell components is sonicated in the presence of the relevant microbubble component gas.

The chemotherapeutic agents herein described may be carried by the microbubble, i.e. attached to or otherwise associated with the microbubble, using various methods including those which are known in the art. For example, these may be linked (e.g. directly or indirectly) to the microbubble via covalent or non-covalent means, e.g. via electrostatic interaction, van der Waals forces and/or hydrogen bonding. Alternatively, these may be encapsulated by the bubble, for example incorporated into the core of the microbubble or into its shell structure. Various methods for attachment of an active agent to a microbubble or for the incorporation of an active agent into a microbubble (e.g. the shell of a microbubble) are known in the art and any known methods may be used. Other methods which may be used include those specifically described herein, though these are not intended to be limiting.

In one embodiment, one or more of the selected chemotherapeutic agents may be attached to the microbubble via strong non-covalent bonding, for example via a biotin-avidin interaction. In this case, one component of the binding pair is functionalised with biotin and the other is functionalised with avidin. For example, the chemotherapeutic may be functionalised with biotin and the microbubble may be functionalised with avidin. Typically, the avidin molecule will also be bound to the microbubble via a biotin-avidin interaction. For example, the microbubble may be functionalised with biotin to form a biotinylated microbubble which is then incubated with avidin. Once the avidin is bound to the microbubble, this permits the binding of any further biotinylated moieties such as the chemotherapeutic agent. The resulting linkage between the microbubble and the chemotherapeutic agent may thus take the form of a “biotin-avidin-biotin” interaction.

In one embodiment, the platinum-based chemotherapeutic or derivative thereof is attached to the microbubble via a non-covalent bond, for example via a biotin-avidin-biotin interaction. For example, oxaliplatin or any derivative of oxaliplatin (such as those herein described) may be biotinylated and linked to the biotinylated shell of the bubble via avidin. In one particular embodiment, Pt(DACH)(Ox)(OH)₂ can be biotinylated and attached to the microbubble in this way.

In one embodiment, the 5-fluoropyrimidine or derivative thereof is attached to the microbubble via a non-covalent bond, for example via a biotin-avidin-biotin interaction. For example, 5-FU may be biotinylated and linked to the biotinylated shell of the bubble via avidin.

In one embodiment, one or more of the selected chemotherapeutic agents may be attached to the microbubble via one or more covalent bonds, e.g. via a single covalent bond. Methods which may be used for covalently attaching a chemotherapeutic agent to a microbubble include known chemical coupling techniques. The exact method used will be dependent on the exact nature of the microbubble and the chemotherapeutic agent, specifically the nature of any pendant functional groups. If necessary, one or both components which are to be linked may be functionalised, e.g. to include reactive functional groups which may be used to couple the molecules. Suitable reactive groups include acid, hydroxy, carbonyl, acid halide, thiol and/or primary amine. Methods for the introduction of such functional groups are well known in the art. Examples of methods which may be used to covalently bind a microbubble to a chemotherapeutic agent include, but are not limited to, the following: a) Carbodiimide based coupling methods. These may be used to couple microbubbles containing either an amine or carboxylic acid functionality to a moiety having either a carboxylic acid or amine functionality. Such methods result in the formation of ester or amide bonds; b) “CLICK” reaction (i.e. 1,3-dipolar cycloaddition reaction). This may be used to react azide or acetylene functionalised microbubbles with a moiety having either acetylene or azide functionality; c) Schiff base formation (i.e. imine bond formation). This reaction may be used to bond aldehyde or amine functionalised microbubbles to a moiety containing amine or aldehyde functionality; and d) Michael addition reactions.

Alternatively, methods for covalent attachment of a chemotherapeutic may involve the formation of a “functionalised lipid” which is covalently bound to the chemotherapeutic before preparation of the microbubble-complex. Such methods may be advantageous over attachment methods which involve non-covalent attachment such as the biotin-avidin-biotin interaction described herein. For example, covalent attachment of the chemotherapeutic agent to the lipid prior to formation of the microbubble avoids the need to manipulate the bubble once it has been produced. By avoiding the need for multiple conjugation steps to be carried out on the bubble once it has been formed, the yield of the microbubble-complex is also improved. In addition to this, pre-treatment of the lipid to introduce the selected chemotherapeutic agent (or agents) allows for greater control over their introduction into the final microbubble-complex. As a result, drug loading levels can be more precisely tailored.

In some cases, the precise nature of the covalent linkage between the chemotherapeutic agent (or agents) and the lipids which form the shell structure of the microbubble may be selected to improve the delivery of the agent(s) to the target cells or tissues. For example, where the lipid is a phospholipid, the chemotherapeutic agent is released to the target cells or tissues in phosphorylated form, which is an active metabolite. This is in contrast to the non-covalent biotin-avidin binding methods in which the chemotherapeutic agent, once released from the microbubble, requires phosphorylation for activation.

Preparation of a “functionalised lipid” involves the step of covalent attachment of the chemotherapeutic agent to a lipid capable of forming a microbubble. By a “lipid capable of forming a microbubble”, it is intended that the lipid should be capable of maintaining a microbubble by forming a layer at the interface between a gas within the core of the microbubble and an external medium, e.g. an aqueous solution which contains the microbubble. As will be understood, a lipid which is “capable of forming a microbubble” is not intended to encompass a lipid which has already been incorporated into the shell structure of a microbubble. Covalent attachment will comprise the formation of at least one covalent linkage between the selected lipid and chemotherapeutic agent, but it may also involve the formation of more than one covalent linkage. Typically, a single lipid will be functionalised by a single covalent linkage to a single chemotherapeutic agent. As will be described herein, covalent linkage of the therapeutic agent and the lipid may be ‘direct’, or it may be ‘indirect’, i.e. via a suitable linking moiety which is covalently attached both to the lipid and to the chemotherapeutic agent.

Lipids and combinations of different lipids (e.g. combinations of two different lipids) may be used to produce the microbubble-complexes. Where more than one type of lipid is used, it will be understood that at least one of the lipids will be modified as herein described to form a “functionalised lipid” carrying at least one (e.g. one) of the selected chemotherapeutic agents via a covalent linkage. However, it is not essential that all lipids used to form the microbubble-complex will be functionalised in this way. Other non-functionalised lipids (i.e. lipids which do not carry any chemotherapeutic agent) may also be used. Such lipids will also be capable of forming a microbubble and may be any of those herein described.

In one embodiment, a proportion of the lipids used to produce the microbubble-complex may be modified to carry one or more biocompatible polymers or polysaccharides, for example a polyalkylene glycol such as polyethylene glycol (PEG). Lipids bearing polymers such as PEG, including but not limited to PEG 2000 MW, PEG 5000 MW, and PEG 8000 MW, are particularly suitable for improving the stability and size distribution of the microbubbles. Different mole ratios of lipids bearing polymers (e.g. a PEGylated lipid) and other lipids may be used. These may be used in combination with one or more functionalised lipids as herein described.

Methods suitable for covalent linkage of the selected lipid and chemotherapeutic agent(s) to produce a functionalised lipid may readily be determined by those skilled in the art. The choice of method to be used will depend on the chemical structure of the lipid and the chemotherapeutic agent, for example on the nature of any pendant functional groups which may undergo a chemical reaction to form the desired covalent bond. Examples of covalent bonds which may be formed include ester, amide, ether, carbamate, urea, thiourea, sulphide, disulfide, sulfone, and carbonate linkages, and C—C bonds. Where appropriate, the lipid and/or the chemotherapeutic agent may be suitably modified to enable their reaction, for example these may be modified to introduce a suitable functional group.

In some embodiments, the lipid in its conventional (e.g. commercially available) form, whether synthetic, semi-synthetic or natural, may possess a functional group capable of linking to the desired therapeutic agent by a covalent bond. The nature of the functional group present in the lipid is not limited. In the case of a phospholipid, for example, the functional group may be amino in phosphatidylethanolamine, hydroxyl in phosphaditylglycerol, and carboxyl in phosphatidylserine.

In other embodiments, the lipid for use in preparation of the microbubble may be capable of undergoing a transesterification or hydrolysis reaction enabling it to form a covalent link with a hydroxyl-carrying chemotherapeutic agent. As will be understood, such a reaction enables direct linkage of the chemotherapeutic agent to the phosphate ester group of the lipid. The resulting functionalised lipid may, for example, be a compound of formula (II), or a pharmaceutically acceptable salt thereof:

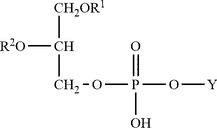

wherein:
R¹ and R², which may be the same or different, are as herein defined; and
Y is the “residue” of a chemotherapeutic agent.

The term “residue” when used in the context of a “residue” of a chemotherapeutic agent refers to the moiety formed when that agent has taken part in a reaction to covalently link it to another compound (e.g. to a lipid or suitably modified lipid) as herein described. Covalent linkage may result from reaction of a terminal group of the chemotherapeutic agent.

In other embodiments, the lipid for use in forming the microbubble may be chemically modified prior to reaction with the selected chemotherapeutic agent. The formation of such a “modified” lipid may be appropriate, for example, where the unmodified lipid is not capable of undergoing a suitable chemical reaction with the chosen chemotherapeutic agent. Modification of the lipid may, for example, alter the functionality of an existing functional group, e.g. the conversion of a carboxylic acid to an amide or ester, etc. Alternatively, the lipid may be modified by reaction with another compound to provide a linking moiety which carries a terminal functional group enabling it to form a covalent link to a chemotherapeutic agent. A non-limiting example of such a modification is the reaction of a lipid with an acid anhydride which provides a linking moiety having a terminal carboxylic acid group. By way of example, a lipid (e.g. a phosphatidylethanolamine) which has been modified in this way may be reacted with a chemotherapeutic agent to provide a “functionalised lipid” of formula (III), or a pharmaceutically acceptable salt thereof:

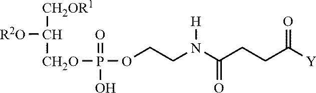

wherein;
R¹ and R², which may be the same or different, are as herein defined; and
Y is the “residue” of a chemotherapeutic agent.

The selected chemotherapeutic agent may be one capable of covalent linkage to the chosen lipid (or suitably modified lipid as herein described) and may, for example, contain one or more groups capable of forming the desired covalent bond with the lipid. If required, however, the chemotherapeutic agent may be suitably “functionalised”, e.g. to include one or more reactive groups which enable its reaction with the lipid (or modified lipid). Any suitable functional groups may be used and these may readily be selected by those skilled in the art. Suitable functional groups which may be introduced include, for example, carboxylic acid, hydroxyl (e.g. primary hydroxyl), carbonyl, acid halide, thiol and/or amine (e.g. primary amine) groups. Methods for the introduction of such functional groups are well known in the art. Functionalisation may involve reaction of the chemotherapeutic agent with a compound which is capable of providing the desired functionality and may result in the introduction of a linking moiety which enables its reaction with the selected lipid. Suitable compounds may readily be determined by any skilled chemist and include, for example, compounds containing terminal amine or carboxylic acid groups. Following reaction with the agent these may, for example, provide a terminal amine or carboxylic acid functionality which is capable of reaction with the chosen lipid.

In one embodiment, a chemotherapeutic agent which comprises a primary hydroxyl group, or which may be modified to introduce such a group, may be covalently linked to the microbubble due to their ease of reaction with a lipid. A particular example of such an agent is 5-FUR.

Where the chosen lipid and/or chemotherapeutic agent are polyfunctional, suitable protecting groups may be used to block the reaction of one or more of the functional groups (e.g. hydroxyl, amine, etc.) to obtain the desired chemoselectivity in the reaction to covalently link the lipid and chemotherapeutic agent. As used herein, a “protecting group” refers to a chemical group which can be introduced into a molecule by chemical modification of a functional group to obtain chemoselectivity in a subsequent chemical reaction. Protecting groups may be introduced onto a specific functional group in a polyfunctional molecule to block its reactivity under reaction conditions needed to make modifications elsewhere in the molecule. Suitable protecting groups should be readily, but selectively, introduced into the desired functional group, be stable to the reagents employed in the subsequent reaction steps and, ideally, be capable of being removed under mild conditions when no longer required. Suitable protecting groups are well known to a person skilled in the art.

Chemical reactions which may be used to covalently link the chosen lipid and chemotherapeutic agent(s) may be determined by those skilled in the art having in mind the nature of the chemical structures of the components to be covalently linked to one another.

Methods for attaching a phospholipid to a compound having a pendant hydroxyl group are, for example, known in the literature. These may involve an enzyme-catalysed reaction between the lipid and chemotherapeutic agent in a biphasic emulsion. For example, Phospholipase D (PLD) is known for use in the hydrolytic conversion of a phospholipid to a phosphatidic acid in the presence of water which can then react with the hydroxyl group of an acceptor. Such transphosphatidylation methods are, for example, described in the following references, the contents of which are incorporated herein by reference: Shuto et al., Chem, Pharm, Bull, 35(1): 447-449, 1987; Shuto et al., Tetrahedron Letters 28(2): 199-202, 1987; Shuto et al., Nucleosides & Nucleotides 11(2-4): 437-446, 1992; Shuto et al., Bioorganic & Medicinal Chemistry 3(3): 235-243, 1995; Shuto et al., Bioorganic & Medicinal Chemistry Letters 6(9): 1021-1024, 1996; and Hirche et al., Enzyme and Microbial Technology 20: 453-461, 1987). The PLD is not subject to any limitations, although that derived from Streptomyces, for example from Streptomyces chromofuscus, from cabbage, or from Arachis hypogaea (peanut), is considered particularly suitable.

Appropriate reaction conditions for enzyme-catalysed transphosphatidylation may readily be determined by those skilled in the art. Typically, the selected lipid, chemotherapeutic agent and enzyme are provided in a two phase system consisting of a suitably buffered aqueous phase and an organic phase. The enzymatic reaction takes place at the phase boundary of the aqueous and organic phase. Suitable divalent metal ions may be required for enzymatic activity dependent on the selected enzyme. If required, these ions may be provided by calcium salts, such as calcium chloride, which are provided in the aqueous buffer. The pH of the aqueous phase will generally range from 3 to 7, preferably from 4 to 6, e.g. it may be about 4.5. The temperature of the reaction may range from about 25° C. to about 50° C., preferably from about 40° C. to about 50° C., e.g. about 45° C. A variety of organic solvents can be used depending on the solubility of the lipid such as, but not limited to, diethyl ether, ethyl acetate, benzene, and hexane.

A non-limiting example of a PLD-catalysed method for covalent attachment of 5-FUR to the lipid DBPC is shown in scheme 1:

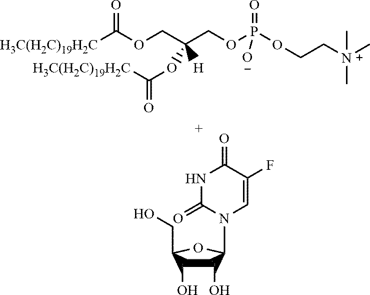

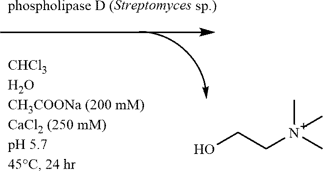

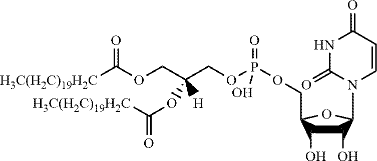

Other non-enzymatic methods may be employed to covalently link the chemotherapeutic agent to a lipid (e.g. a phospholipid) to provide the desired “functionalised lipid”. In one embodiment of such methods, the lipid may be reacted with another compound (e.g. an acid anhydride) to provide a modified lipid prior to covalent linkage to the selected chemotherapeutic agent. For example, where the lipid is a phosphatidylethanolamine, the method for preparing the “functionalised lipid” may be illustrated by way of the following general reaction schemes to produce a “functionalised lipid” of formula (IV) or (V), or a pharmaceutically acceptable salt thereof:

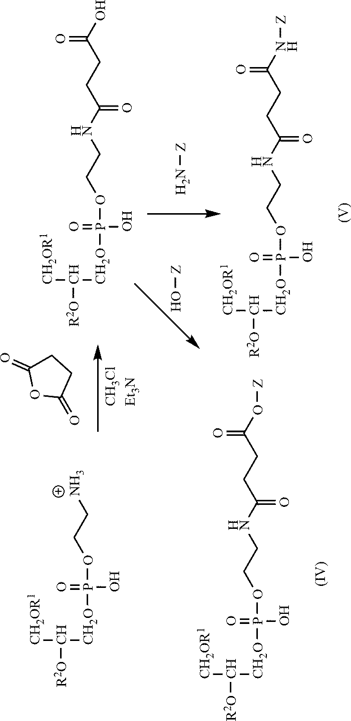

wherein:
R¹ and R², which may be the same or different, are as herein defined; and
Z is the “residue” of a chemotherapeutic agent.

In these reactions, the chemotherapeutic agent (HO-Z or H₂N-Z) may be employed in the form of a suitably protected derivative thereof in order to direct its point of covalent linkage to the modified lipid. Where any protecting groups are employed, it will be understood that the final step of the process to produce the functionalised lipids will typically involve the removal of the protecting group(s).

A non-limiting example of a non-enzymatic method for covalent attachment of an oxaliplatin derivative to the lipid DSPE is shown in scheme 3:

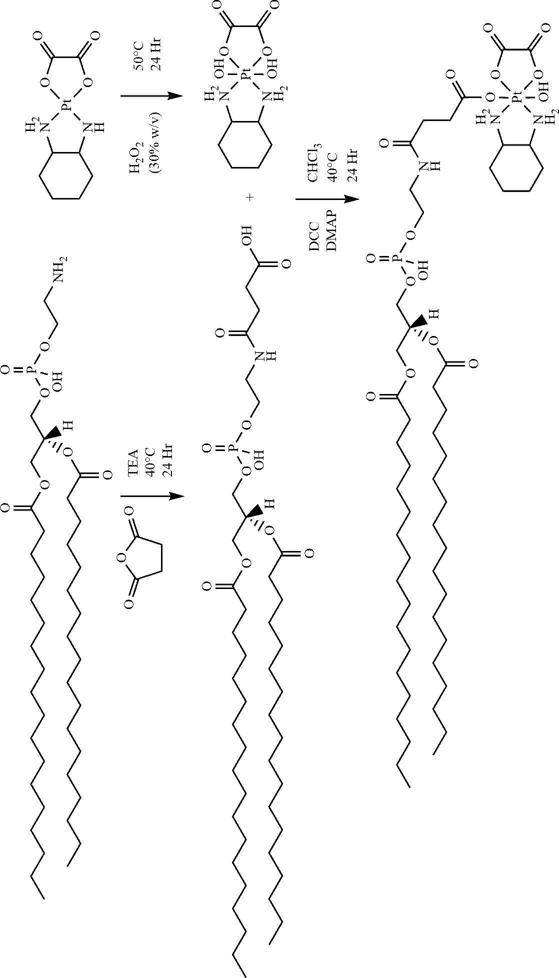

In another embodiment, a non-enzymatic method may involve reaction of the selected chemotherapeutic agent with another compound (e.g. an acid anhydride) to provide a “modified” chemotherapeutic agent prior to covalent linkage to the lipid. For example, where the lipid is a phosphatidylethanolamine, the method for preparing the “functionalised lipid” is illustrated by way of the following general reaction scheme to produce a “functionalised lipid” of formula (VI), or a pharmaceutically acceptable salt thereof:

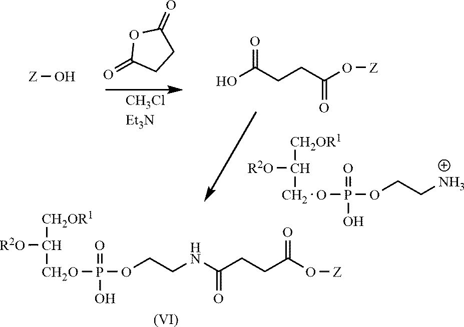

wherein:
R¹ and R², which may be the same or different, are as herein defined; and
Z is the “residue” of a chemotherapeutic agent.

Non-limiting examples of such methods for the covalent attachment of 5-FUR or irinotecan to the lipid DSPE are shown in schemes 5 and 6, respectively. In the method shown in scheme 5, the secondary alcohols of 5-FUR are suitably protected prior to its reaction with succinic anhydride. The resulting “functionalised” 5-FUR is then reacted with the amine of DSPE, followed by deprotection.

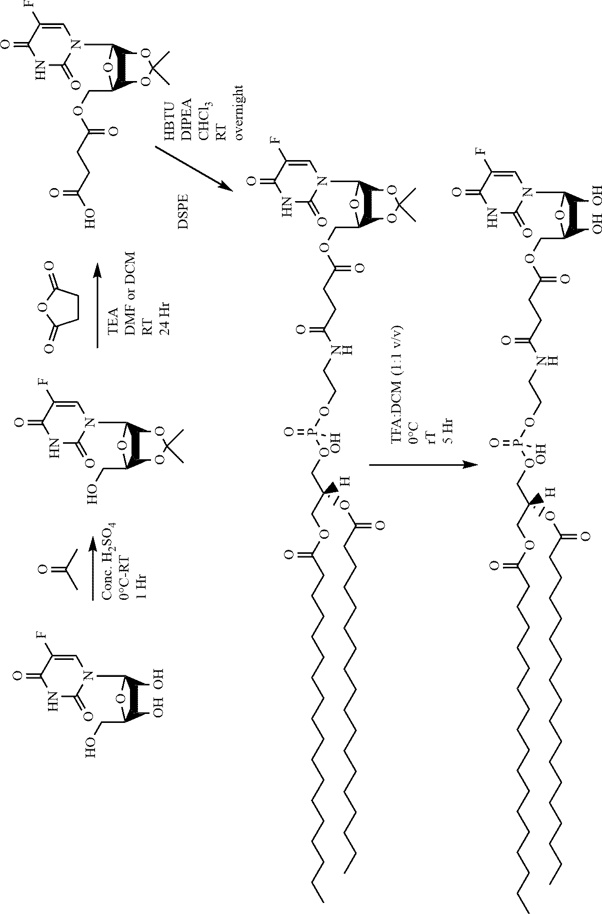

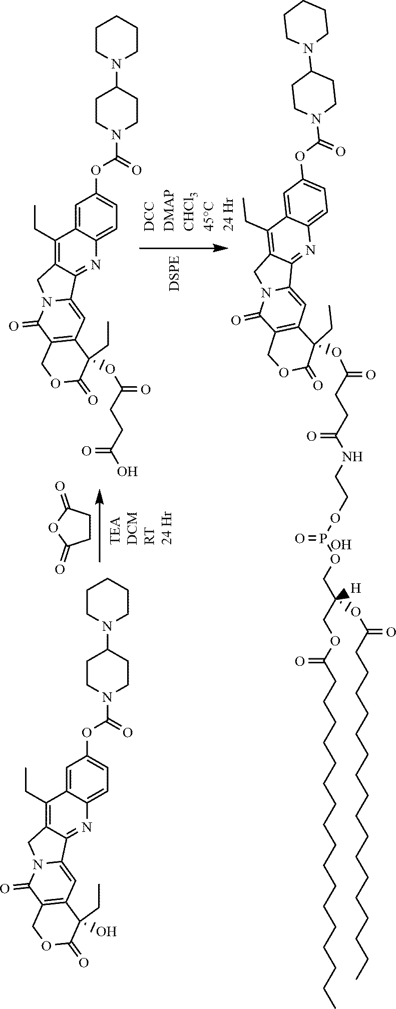

Any of the intermediates formed in any of the methods herein described are also considered to form part of the invention, as are any of the methods used for their preparation.

Any of the methods generally described herein for the formation of a microbubble may be used to convert any “functionalised lipid” into the desired microbubble-complex for use in the invention. Typically such methods will include dispersion of the selected gas in an aqueous suspension which contains the functionalised lipid(s). Techniques which may be used to form the microbubble from this suspension include sonication, mechanical agitation (e.g. high speed mixing), coaxial electrohydrodynamic atomisation and microfluidic processing using a T-junction (see e.g. Stride & Edirisinghe, Med. Biol. Eng. Comput., 47: 883-892, 2009). Mechanical agitation (e.g. high speed mixing) and sonication techniques are generally preferred, in particular high speed mixing methods.

For example, an aqueous suspension comprising the functionalised lipids and containing one or more stabilising agents may provide a suitable particulate suspension. Other non-functionalised lipids, or other lipids bearing polymer groups as herein described, may also be present. Examples of stabilising agents include, but are not limited to, glycerol, cetyl alcohol, sorbitol, polyvinylalcohol, polypropylene glycol, and propylene glycol. In one embodiment a mixture of glycerol and propylene glycol may be used. Solvent systems suitable for the suspension of the lipids may readily be selected. A preferred solvent system may include saline (e.g. phosphate buffered saline), glycerol and propylene glycol, for example in a ratio of 8:1:1. Agitation of the resulting suspension of lipids in the presence of the selected gas produces a stable suspension of gas-filled microbubbles which, if desired, can then be separated from the solution. Agitation of the suspension must involve sufficient force that the gas is introduced into the aqueous solution and to allow the formation of the microbubbles. Typically, agitation will involve high speed mixing or sonication.

Sonication may be carried out using an ultrasound transmitting probe. For example, the aqueous suspension of the lipid(s) may be sonicated in the presence of the relevant microbubble component gas to produce the microbubble-complex. Where sonication is employed, the required duration of sonication may be determined by detection of the formation of the gas-filled microbubbles, for example the formation of a milky-white suspension. In one embodiment, more than one sonication cycle may be performed. For example, two sonication cycles may be carried out. The first cycle may involve sonicating the suspension to fully disperse the lipids and will generally be carried out at low power (e.g. amplitude setting about 20%) with the probe tip of the sonicator fully submerged in the liquid for about 30 seconds. The second cycle may involve sonicating the lipid suspension at a higher power (e.g. amplitude setting about 90%) with the probe tip at the gas-liquid interface and under a headspace of the selected gas (e.g. PFB) for about 30 seconds. The frequency of the probe sonicator may suitably be set at about 20 KHz.

Mechanical agitation, for example by high speed mixing, of the lipid-containing suspension may also be employed to produce the desired gas-filled microbubbles. Suitable shaking frequencies and duration may readily be selected by those skilled in the art. The formation of a milky-white suspension may be taken as an indication of the formation of the desired gas-filled microbubbles. A shaking frequency of about 4530 ± 100 oscillations per minute and/or a shaking duration of about 45 seconds may, for example, be employed.

The concentration of lipid required to form the microbubbles will vary depending on the type of lipid used, but may readily be determined by those skilled in the art. For example, in preferred embodiments, the concentration of lipid used to form the gas-filled microbubbles may be about 2.0 mmol/L, e.g. about 2.2 mmol/L, based on the amount of saline solution.

In one embodiment, the functionalised lipids may be dissolved in an organic solvent which is then evaporated to produce a dried lipid film prior to their conversion to the microbubble-complex. The dried lipid film may be reconstituted in a suitable solvent prior to agitation (e.g. sonication or high speed mixing) to produce the loaded microbubbles. Reconstitution of the dried lipids in a suitable aqueous solvent prior to mixing with the gas ensures that the lipids are introduced into an aqueous solution. The step of reconstitution may involve heating of the aqueous solution above the lipid transition temperature with gentle stirring. For storage prior to use, the loaded microbubbles may be suspended in an aqueous solution, such as a saline (e.g. phosphate buffered saline) solution.

In an alternative method to prepare the microbubbles, an aqueous suspension comprising the functionalised lipids may be lyophilised, for example using a suitable cryoprotectant. The resulting powder can then be reconstituted in a suitable aqueous medium in the presence of the selected microbubble component gas. Reconstitution may, for example, be carried out at the point of use. Such methods involving the formation of a lyophilised powder include those described in US 5,686,060, the contents of which are incorporated herein by reference.

5-FUR is particularly suitable for covalent attachment to a lipid since this carries a pendant primary hydroxyl group enabling this to be readily linked to a lipid without further modification, if desired.

In one embodiment, the 5-fluoropyrimidine chemotherapeutic or derivative thereof is covalently attached to the microbubble. For example, 5-FUR or 5-FU may be covalently attached.

In one embodiment, the platinum-based chemotherapeutic or derivative thereof is covalently attached to the microbubble. For example oxaliplatin or a derivative thereof, such as Pt(DACH)(Ox)(OH)₂, is covalently attached.

In another embodiment, both the 5-fluoropyrimidine chemotherapeutic or a derivative thereof, and the platinum-based chemotherapeutic or a derivative thereof are covalently attached to the microbubble.

In one embodiment, irinotecan or a derivative thereof is covalently attached to the microbubble.

In one embodiment, all chemotherapeutic agents are covalently attached to the microbubble.

Any hydrophobic chemotherapeutic agent may be incorporated within the shell structure of the microbubble. For example, the microbubble may comprise a shell having incorporated therein one or more of the selected chemotherapeutic agents. In this case, the chemotherapeutic agent should be capable of spontaneously embedding within the hydrophobic layer of the microbubble, e.g. within the hydrophobic lipid chains of the microbubble lipids. In this case, the association between the chemotherapeutic agent and the bubble is a direct hydrophobic interaction. If required, the hydrophobic tail region of the lipids may be suitably modified to carry a polymer, such as PEG (e.g. PEG-2000). A hydrophobic agent may be considered to be one having a LogP value greater than about 2. Alternatively, any non-hydrophobic chemotherapeutic agent may be suitably modified (e.g. functionalised) by the introduction or one or more non-polar functional groups which enable it to spontaneously embed within the shell (e.g. the lipid shell) of the microbubble.

In the case where a chemotherapeutic is to be incorporated within the shell of the microbubble this may, for example, be dissolved in an organic solvent and added to a solution containing the lipids (e.g. functionalised lipids) prior to formation of the microbubble-complex as described herein. During microbubble formation, the chemotherapeutic agent distributes into the hydrophobic shell structure of the microbubble-complex.

In one embodiment, irinotecan or a derivative of irinotecan is embedded within the shell of the microbubble. When embedded in the shell of the microbubble as herein described, irinotecan will typically be used in the form of its free base.

In other embodiments, any of the chemotherapeutic agents herein described can be attached to the microbubble in the form of a liposomal complex having the agent embedded therein. The chemotherapeutic agent may be hydrophilic or hydrophobic. In these embodiments, the drug-loaded liposome can be attached to the shell of the microbubble using any of the covalent or non-covalent methods herein described whereby to provide a “microbubble-liposome conjugate”. In one embodiment, a drug-loaded liposome may be conjugated to the microbubble using a biotin-avidin-biotin crosslink.

The use of a liposome encapsulating a chemotherapeutic agent within its aqueous core is particularly suitable for loading any hydrophilic chemotherapeutic agent onto the microbubble. Irinotecan, for example, is sold as an aqueous solution under the tradename Camptosar® containing the active in the form of its hydrochloride salt. In one embodiment, the hydrochloride salt of irinotecan may be loaded onto the microbubble in liposomal form. A liposomal form of irinotecan which may be used is that sold under the tradename Onivyde™. Oxaliplatin is also hydrophilic and may, for example, be incorporated within a liposome for attachment to the shell of the bubble.

Methods for the preparation of liposomes carrying active agents are well known in the art and include methods similar to those herein described for preparation of the microbubbles. In one embodiment, a mixture of lipids may be dissolved in an organic solvent which is then evaporated to produce a dried lipid film. Where the intention is to encapsulate a hydrophobic drug within the liposome, this will typically be incorporated into the lipid mixture prior to solvent evaporation. The dried lipid film can then be reconstituted in a suitable solvent. Where the intention is to encapsulate a hydrophilic drug within the liposome, an aqueous solution of the selected hydrophilic drug can be used to form a suspension of multilamellar vesicles. Agitation of the mixture, for example by sonication, produces the desired drug-loaded liposomes. In the case where the liposome is intended to be linked to the bubble via a biotin-avidin-biotin interaction, a proportion of the lipids will be employed in biotinylated form. Avidin may then be added to a suspension of a biotinylated microbubble. Following the removal of excess avidin, a suspension of the drug-loaded biotinylated liposome may then be combined with the microbubble suspension and mixed to produce the loaded microbubbles. For storage prior to use, the liposome-loaded microbubbles may be suspended in an aqueous solution, such as a saline (e.g. PBS).

The drug-loaded microbubbles (i.e. “microbubble-complexes”) herein disclosed form a further aspect of the invention. In another aspect, the invention also provides any microbubble-complex obtained or obtainable by any of the methods herein disclosed.

Any of the methods herein disclosed for preparation of the microbubble-complexes also form part of the invention. It will be understood that any of the present disclosure relating to the selected chemotherapeutics, the lipids or functionalised lipids which form the microbubbles, etc., also extends to the microbubble-complexes according to the invention and methods for their preparation.

In a further aspect, the invention thus provides a microbubble-chemotherapeutic agent complex comprising a microbubble carrying a combination of the following chemotherapeutic agents:
(a) a 5-fluoropyrimidine or a derivative thereof;
(b) irinotecan or a derivative thereof; and
(c) a platinum-based chemotherapeutic agent or a derivative thereof.

The selected chemotherapeutic agents may be carried by the microbubble, i.e. attached to or otherwise associated with the microbubble, using any of the methods herein described. In one embodiment, the 5-fluoropyrimidine or derivative will be covalently linked to the microbubble. In one embodiment, irinotecan in the form of its free base will be embedded within the shell of the microbubble. In one embodiment, the platinum-based chemotherapeutic or derivative will be non-covalently linked to the microbubble, e.g. via a biotin-avidin-biotin interaction. In another embodiment, the platinum-based chemotherapeutic or derivative will be covalently linked to the microbubble.

In one embodiment, the microbubble may be covalently linked to the 5-fluoropyrimidine or derivative, non-covalently linked to the platinum-based chemotherapeutic or derivative (e.g. linked via a biotin-avidin-biotin interaction), and will have a shell in which irinotecan or a derivative thereof is embedded.

In another embodiment, the microbubble may be covalently linked both to the 5-fluoropyrimidine or derivative and to the platinum-based chemotherapeutic or derivative, and will have a shell in which irinotecan or a derivative is embedded.

In another aspect, the invention provides a method for the preparation of a microbubble-chemotherapeutic agent complex as herein described, wherein said method comprises the following steps:
(i) providing a lipid which is capable of forming a microbubble;
(ii) optionally covalently linking one or more chemotherapeutic agents to said lipid whereby to form a functionalised lipid;
(iii) preparing a microbubble from said functionalised lipid, optionally in the presence of one or more chemotherapeutic agents; and
(iv) optionally linking the resulting microbubble to one or more chemotherapeutic agents via a non-covalent linkage, e.g. via a biotin-avidin-biotin interaction.

In one embodiment, the method comprises:
(i) providing a lipid which is capable of forming a microbubble;
(ii) covalently linking a 5-fluoropyrimidine or derivative thereof to said lipid whereby to form a functionalised lipid;
(iii) preparing a microbubble from said functionalised lipid in the presence of irinotecan; and
(iv) linking the resulting microbubble to a platinum-based chemotherapeutic.or derivative thereof via a biotin-avidin-biotin interaction.

In another embodiment, the method comprises:
(i) providing a first lipid which is capable of forming a microbubble;
(ii) covalently linking said 5-fluoropyrimidine or derivative thereof to said first lipid whereby to form a first functionalised lipid;
(iii) providing a second lipid which is capable of forming a microbubble;
(iv) covalently linking said platinum-based chemotherapeutic or derivative thereof to said second lipid whereby to form a second functionalised lipid; and
(v) preparing a microbubble from a mixture of said first and said second functionalised lipids in the presence of irinotecan.

The microbubble-chemotherapeutic agent complexes herein described find use in methods of medical treatment, in particular in the treatment of cancer. In one aspect, the invention thus provides a microbubble-chemotherapeutic agent complex as herein described for use in therapy or for use as a medicament.

As used herein, the term “cancer” refers to cells undergoing abnormal proliferation. Growth of such cells typically causes the formation of a tumour. Cancerous cells may be benign, pre-malignant or malignant. Such cells may be invasive and/or have the ability to metastasize to other locations in the body. The term cancer, as used herein, includes cancerous growths, tumours, and their metastases. The term “tumour”, as used herein, refers to an abnormal mass of tissue containing cancerous cells. As used herein, the term “metastasis” refers to the spread of malignant tumour cells from one organ or part of the body to another non-adjacent organ or part of the body. Cancer cells may break away from a primary tumour, enter the lymphatic and blood systems and circulate to other parts of the body (e.g. to normal tissues). Here they may settle and grow within the normal tissues. When tumour cells metastasize, the new tumours may be referred to as a “secondary” or metastatic cancer or tumour. The term “metastatic disease” as referred to herein relates to any disease associated with metastasis.

As used herein, “treatment” includes any therapeutic application that can benefit a human patient. Treatment is intended to refer to the reduction, alleviation or elimination, of a disease, condition or disorder. It includes palliative treatment, i.e. treatment intended to minimise, or partially or completely inhibit the development of the disease, condition or disorder. The term “patient”, as used herein, refers to a human subject under the treatment of a clinician.

The methods of treatment herein described find particular use in the treatment of tumours such as sarcomas and carcinomas, in particular solid tumours. The invention is particularly suitable for the treatment of solid tumours which are located below the surface of the skin.

Non-limiting examples of tumours that may be treated using the methods herein described are sarcomas, including osteogenic and soft tissue sarcomas; carcinomas, e.g. breast, lung, cerebral, bladder, thyroid, prostate, colon, rectum, pancreas, stomach, liver, uterine, hepatic, renal, prostate, cervical and ovarian carcinomas; lymphomas, including Hodgkin and non-Hodgkin lymphomas; neuroblastoma, melanoma, myeloma, Wilm’s tumour; leukemias, including acute lymphoblastic leukaemia and acute myeloblastic leukaemia; astrocytomas, gliomas and retinoblastomas. In particular, the following tumours and any associated metastatic condition may be treated: pancreatic cancer, breast cancer, prostate cancer, glioma, non-small cell lung carcinoma, head and neck cancers, cancers of the urinary tract, kidney or bladder, advanced melanoma, oesophageal cancer, colon cancer, hepatic cancer, and lymphoma. The treatment of pancreatic and colon cancers, and their associated metastases, forms a preferred aspect of the invention. In one embodiment, the methods herein described can be used to treat pancreatic adenocarcinoma (PAC) or metastatic pancreatic adenocarcinoma (mPAC).

For use in any of the methods of treatment herein described, the microbubble-complex carrying the chemotherapeutics will generally be provided in a pharmaceutical composition together with at least one pharmaceutically acceptable carrier or excipient. Such pharmaceutical compositions form a further aspect of the invention. By “a pharmaceutical composition” is meant a composition in any form suitable to be used for a medical purpose. In a further aspect, the invention thus provides a pharmaceutical composition comprising a microbubble-complex as herein described together with at least one pharmaceutically acceptable carrier or excipient.

Suitable pharmaceutical compositions may be formulated using techniques well known in the art. Their route of administration will depend on the intended use. Typically, these will be administered systemically and may thus be provided in a form adapted for parenteral administration, e.g. by intradermal, subcutaneous, intraperitoneal or intravenous injection. Suitable pharmaceutical forms thus include, but are not limited to, suspensions and solutions which contain the microbubble-complex together with one or more inert carriers or excipients. Suitable carriers include saline, sterile water, phosphate buffered saline and mixtures thereof. The compositions may additionally include other agents such as emulsifiers, suspending agents, dispersing agents, solubilisers, stabilisers, buffering agents, wetting agents, preserving agents, etc. The compositions may be sterilised by conventional sterilisation techniques. Solutions containing the microbubble-complex may be stabilised, for example by the addition of agents such as viscosity modifiers, emulsifiers, solubilising agents, etc. Typically, the pharmaceutical compositions will be used in the form of an aqueous suspension of the microbubble-complex in water or a saline solution, e.g. phosphate-buffered saline. The microbubble-complex may be supplied in the form of a lyophilised powder for reconstitution at the point of use, e.g. for reconstitution in water, saline or PBS.

For use in the treatment of cancer, the microbubble-complex herein described is administered in combination with folinic acid or a derivative thereof. The combined use of folinic acid or a derivative of folinic acid serves to modulate the activity of the 5-fluoropyrimidine chemotherapeutic and/or reduce its side effects. Since the folinic acid is not toxic, it need not be delivered on a microbubble and may be administered separately, simultaneously or sequentially with the microbubble-complex. As will be understood, it will be administered in such a way that it is present at the target tissue at the point of rupture of the microbubbles and release of the chemotherapeutic agents. The folinic acid (or derivative) will typically be administered via its conventional route, i.e. intravenous injection. A suitable dosage can readily be selected by those skilled in the art.

The microbubble-complex and folinic acid or derivative thereof may be administered to the subject separately, simultaneously or sequentially. In an embodiment, the microbubble-complex and folinic acid (or derivative) will be administered separately from one another, preferably sequentially. Where they are administered sequentially, they may be administered in either order. In one embodiment, the folinic acid or folinic acid derivative is administered prior to administration of the microbubble-complex. For example, it may be administered up to several hours prior to administration of the microbubble-complex. In particular, the folinic acid or derivative may be administered up to 1 hour, e.g. up to about 30 minutes, before the microbubble-complex is administered.

In another embodiment, the microbubble-complex and folinic acid (or derivative) may be administered simultaneously. For example, the microbubble-complex and folinic acid (or derivative) may be co-administered in a single pharmaceutical preparation, e.g. an aqueous solution. However, in another embodiment these may be administered separately (e.g. either simultaneously or sequentially) in separate pharmaceutical formulations.

In another aspect, the invention thus provides a pharmaceutical composition comprising a microbubble-complex as herein described and folinic acid or a derivative thereof, together with at least one pharmaceutical carrier or excipient.

In another aspect the invention provides the use of a microbubble-complex as herein described in the manufacture of a medicament for use in combination therapy with folinic acid or a derivative thereof, e.g. in a method of treatment of cancer.

In another aspect the invention provides the use of folinic acid or a derivative thereof in the manufacture of a medicament for use in combination therapy with a microbubble-complex as herein described, e.g. in a method of treating cancer.

Corresponding methods of medical treatment also form an aspect of the invention. In another aspect, the invention thus provides a method of treating cancer in a patient in need thereof, said method comprising the steps of administering to affected cells or tissues of said patient an effective amount of a microbubble-complex as herein described; simultaneously, separately or sequentially administering to said patient an effective amount of folinic acid or a derivative thereof; and subjecting said target cells or tissues to ultrasound irradiation whereby to rupture said microbubble.

In another aspect the invention provides a product comprising a microbubble-complex as herein described and folinic acid or a derivative thereof for simultaneous or separate use in a method of treatment of cancer.

In another aspect, the invention provides a kit (or pharmaceutical pack) comprising the following components: (i) a microbubble-complex as herein described; and separately (ii) folinic acid or a derivative thereof; optionally together with instructions for use of the components of the kit in a method as herein described. When used, the components of the kit may be administered simultaneously, separately or sequentially. In one embodiment, component (i) may be provided in dry form, e.g. as a lyophilised powder. In this case, the kit may also comprise a container containing a sterile, physiologically acceptable liquid for reconstitution of the powdered form, e.g. saline or PBS, and optionally a gas (e.g. oxygen or a perfluorocarbon).

The methods herein described involve administration of a pharmaceutically effective amount of the microbubble-complex or a composition containing the microbubble-complex. The microbubble-complex releases its drug payload when subjected to ultrasound irradiation which is capable of rupturing the bubble. Exposure of the target area in the body to ultrasound will be carried out during administration of the composition which contains the microbubble-complex. Preferably, exposure to ultrasound will be carried out both prior to and during administration of the composition. Where the half-life of the microbubble-complex is low, this can avoid the situation in which a significant proportion may be removed before the target area receives the ultrasound.

As used herein, a “pharmaceutically effective amount” relates to an amount that will lead to the desired pharmacological and/or therapeutic effect, i.e. an amount which is effective to achieve its intended purpose. While the needs of any individual patient may vary, determination of optimal ranges for effective amounts of the active agent(s) herein described is within the capability of one skilled in the art. Generally, the dosage regimen may be selected by those skilled in the art in accordance with a variety of factors including the nature of the condition and its severity. The effective dose of any of the compositions herein described will depend on the nature of the microbubble-complex, the mode of administration, the condition to be treated, the patient, etc. and may be adjusted accordingly.

The inventors’ findings relating to improvements in tumour growth delay when using low doses of the chemotherapeutics leads to the possibility of using doses of these that are lower than those used in conventional treatments. In some embodiments, the methods herein described will comprise administration to the patient of a sub-therapeutic dosage of one or more, preferably all, of the selected chemotherapeutic agents. As used herein, the term “sub-therapeutic” refers to a dose of the agent which, when used in the standard treatment, would elicit little or no positive effect in the intended treatment, for example, a dose having a non-statistically significant effect on tumour growth. As will be understood, any reference herein to a “standard treatment” refers to the combined use / administration of the selected chemotherapeutic agents in non-microbubble form. The dose of each chemotherapeutic in a standard treatment will vary depending on various factors, such as the type and extent of the cancer, the performance status of the patient, etc., but will typically be dictated by a standard treatment protocol, for example one set by the European Society for Medical Oncology (ESMO). In the treatment of pancreatic cancer, for example, the following may be considered the “standard treatment” for the purposes of the present disclosure:

| | | | |
|---|---|---|---|
| Drug | Dose | Route | Frequency |
| Oxaliplatin | 85 mg/m² | intravenous | Day 1 of 14 day cycle |
| Folinic acid | 350 mg | intravenous | Day 1 of 14 day cycle |
| Irinotecan | 180 mg/m² | intravenous | Day 1 of 14 day cycle |
| Fluorouracil (optional) | 400 mg/m² | Intravenous bolus injection | Day 1 of 14 day cycle |
| Fluorouracil | 2400 mg/m² | intravenous | Days 1 and 2 of 14 day cycle |

In certain embodiments, the dose of one or more of the chemotherapeutic agents for use in a single treatment cycle according to the invention (for example, in a single treatment cycle of 14 days) will be reduced compared to the standard treatment. In one set of embodiments, the dose of all chemotherapeutic agents will be reduced compared to the standard treatment in any given treatment cycle.

In certain embodiments, the 5-fluoropyrimidine or derivative thereof may be administered at a dose of less than 4800 mg/m² per cycle, preferably at a dose not greater than 2400 mg/m² (e.g. per 14 day cycle of treatment). In certain embodiments, irinotecan or any derivative thereof may be administered at a dose of less than 180 mg/m² per cycle, preferably at a dose not greater than 90 mg/m² (e.g. per 14 day cycle of treatment). In certain embodiments, the platinum-based chemotherapeutic agent or derivative thereof may be administered at a dose of less than 85 mg/m² per cycle, preferably at a dose not greater than 42.5 mg/m² (e.g. per 14 day cycle of treatment).

As will be understood, the dosage of folinic acid (or any derivative of folinic acid) may be adjusted accordingly based on the reduced dose of the 5-fluoropyrimidine or derivative thereof.

In one embodiment, the following dosages of the chemotherapeutic agents may be given in a single treatment cycle, for example in a single treatment cycle of 14 days: a 5-fluoropyrimidine or derivative (e.g. 5-FU or 5-FUR): not greater than 2400 mg/m²; irinotecan or a derivative thereof: not greater than 90 mg/m²; and a platinum-based chemotherapeutic agent or derivative thereof (e.g. oxaliplatin or Pt(DACH)(Ox)(OH)₂): not greater than 42.5 mg/m².

The ability to employ significantly reduced doses, in particular sub-therapeutic doses, of the chemotherapeutic agents in the treatment reduces its toxicity. This provides a significant advance in the use of the FOLFIRINOX treatment for pancreatic cancer in that it potentially allows to increase cycle frequency. It also has the potential to open up the treatment to patients who would otherwise not be considered suitable for “standard” FOLFIRINOX.

The European Society for Medical Oncology (ESMO) guidelines only recommend the use of standard FOLFIRINOX treatment in those patients who are otherwise fit and healthy, i.e. who have a good Eastern Cooperative Oncology Group (ECOG) performance status (PS). PS according to ECOG are scales and criteria used by physicians to assess how a patient’s disease is progressing, to assess how the disease affects the daily living abilities of the patients and determine appropriate treatment and prognosis. Performance status 1 identifies “patients restricted in physically strenuous activity but ambulatory and able to carry out work of a light or sedentary nature, e.g. light housework, office work”. Performance status 2 identifies “ambulatory patients capable of all self-care but unable to carry out any work activities, up and about more than 50% of waking hours”. Performance status 3 identifies “patients capable of only limited self-care, confined to bed or chair more than 50% of waking hours. Performance status 4 identifies “patients who are completely disabled, cannot carry on any self-care, totally confined to bed or chair”.

The methods herein described find particular use in the treatment of patients who would not otherwise be eligible for FOLFIRINOX treatment under existing treatment guidelines. For example, these find use in the treatment of patients whose ECOG performance status (ECOG PS) is > 1. In particular, patients having an ECOG PS of 2 or greater, e.g. an ECOG PS of 2 or 3 may be suitable for treatment. In one embodiment, ECOG PS 2 patients may receive the treatment herein described.

In a conventional course of treatment for pancreatic cancer, an initial “full dose” FOLFIRINOX treatment will often be followed by one or more additional “dose-modified” FOLFIRINOX treatments in which the dose is reduced. That compromises the efficacy of the treatment. Due to the reduced toxicity associated with the treatment herein described, however, no such reduction in dose is required and further courses of treatment need not be “dose-modified”. In another embodiment, the treatment methods herein described may thus involve multiple cycles of chemotherapy without any reduction in the dose of chemotherapeutic agents.

Due to its extreme toxicity, conventional FOLFIRINOX is not generally recommended as a second-line treatment in cancer therapy. The aim of any second-line treatment is not only the effectiveness in cancer treatment but also a safe and low toxicity profile for the patient. A patient’s tolerability to a further line of treatment is generally worse after first-line chemotherapy. However, the reduced toxicity of the treatment herein described means that this may be suitable for use as a second-line treatment. As referred to herein, a “second-line treatment” is understood to be a treatment for a disease or condition which is carried out after an initial (i.e. first-line) treatment as failed or stopped working.

As a result of the reduced toxicity of the treatment herein described, the invention also finds use in the treatment of patients who suffer from hepatic or renal dysfunction.

The frequency and intensity of the ultrasound which may be used in any of the treatment methods herein described can be selected based on the need to achieve selective destruction of the microbubble at the target site and may, for example, be matched to the resonant frequency of the microbubble. Ultrasound frequencies will typically be in the range 20 kHz to 10 MHz, preferably 0.1 to 2 MHz. Ultrasound may be delivered as either a single frequency or a combination of different frequencies. Intensity (i.e. power density) of the ultrasound may range from about 0.1 W/cm² to about 1 kW/cm², preferably from about 1 to about 50 W/cm². Treatment times will typically be in the range of 1 ms to 20 minutes and this will be dependent on the intensity chosen, i.e. for a low ultrasound intensity the treatment time will be prolonged and for a higher ultrasound intensity the treatment time will be lower. Ultrasound may be applied in continuous or pulsed mode and may be either focused or delivered as a columnar beam. Any radiation source capable of producing acoustic energy (e.g. ultrasound) may be used in the methods herein described. The source should be capable of directing the energy to the target site and may include, for example, a probe or device capable of directing energy to the target tissue from the surface of the body.

In the methods herein described, the application of ultrasound ruptures the microbubble and releases its payload at the target site. It is not intended that the drug-loaded microbubbles should be used in any method of sonodynamic therapy in which ultrasound is used to activate a sonosensitising agent to generate reactive oxygen species, such as singlet oxygen. The microbubble-complexes for use in the invention thus do not carry any sonosensitising agent. The term “sonosensitising agent” refers to any compound capable of converting acoustic energy (e.g. ultrasound) into reactive oxygen species that result in cell toxicity.

In certain embodiments, any of the therapeutic methods herein described may also include simultaneous, separate or sequential administration of an immune checkpoint inhibitor. Such methods find particular use in the treatment of metastatic disease, for example.

Immune checkpoints are well known in the art and the term is well understood in the context of cancer therapy. Perhaps the most well known are PD-1 and its ligand PDL-1, and CTLA-4. Others include OX40, TIM-3, KIR, LAG-3, VISTA and BTLA. Inhibitors of immune checkpoints, herein generally referred to as “immune checkpoint inhibitors”, inhibit their normal immunosuppressive function, for example by down regulation of expression of the checkpoint molecules or by binding thereto and blocking normal receptor / ligand interactions. As the immune checkpoints put the brakes on the immune system response to an antigen, so an inhibitor thereof (i.e. an “immune checkpoint inhibitor”) reduces this immunosuppressive effect and enhances the immune response.

Any compound capable of inhibiting the normal immunosuppressive function of an immune checkpoint may be used as an “immune checkpoint inhibitor”. In one embodiment, the immune checkpoint inhibitor is an antibody that binds to a specific immune checkpoint molecule whether that immune checkpoint molecule is itself a receptor or a ligand therefor. Receptors which form part of an immune checkpoint are typically found on the surface of T-cells. Those skilled in the art can readily determine agents which may function as an inhibitor of a specific immune checkpoint target. Suitable inhibitors may, for example, be selected from the group consisting of proteins, peptides, peptidomimetics, peptoids, antibodies, antibody fragments, small inorganic molecules, small non-nucleic acid organic molecules or nucleic acids such as anti-sense nucleic acids, small interfering RNA (siRNA) molecules, oligonucleotides, and any combination thereof. The inhibitor may, for example, act to down regulate expression of an immune checkpoint molecule. The inhibitor may, for example, be a modified version of the natural ligand, such as a truncated version of one of the ligands. It may be naturally occurring, recombinant or synthetic. In one embodiment, the immune checkpoint inhibitor may be an antibody which inhibits a particular immune checkpoint molecule. Inhibitors of cytotoxic T-lymphocyte-associated antigen-4 (CTLA-4), programmed cell death-1 (PD-1) and its ligand, PDL-1, are preferred, for example antibodies thereto.

Immune checkpoint inhibitors which may be used in the invention include, but are not limited to, inhibitors of PD-1, PDL-1, CTLA-4, LAG-3 (Lymphocyte Activation Gene-3) and TIM-3 (T-cell Immunoglobulin Mucin-3). In one embodiment, the immune checkpoint inhibitor is a PD-1 inhibitor, a PDL-1 inhibitor, or a CTLA-4 inhibitor. Examples of such drugs are known and used in the art and any may be suitable for use in the invention. Examples of PD-1 inhibitors which may be used in the invention include, but are not limited to, nivolumab (Opdivo), pembrolizumab (Keytruda), spartalizumab, TSR-042, atezolizumab (MPDL3280A), avelumab, and duravlumab. Other examples include BMS-1001 and BMS-1166 developed by BMS (see Skalniak et al., Oncotarget, 2017; 8(42): 72167-72181, the entire content of which is incorporated herein by reference), and SB415286 (see Taylor et al., Cancer Res. 2018, 78(3), 706-717, the entire content of which is incorporated herein by reference). Non-limiting examples of CTLA-4 inhibitors which may be used in the invention include ipilimumab (Yervoy), and tremelimumab. Any combination of known immune checkpoint inhibitors may also be used in the invention.

Loading of the selected chemotherapeutic agents as described herein onto a single microbubble offers additional advantages, for example in terms of the ease of preparation of the complex. It also provides for a high degree of control over the introduction of the agents onto the bubble, i.e. control over the drug-loading levels and thus accurate dosages. However, it is envisaged that a combination of separate microbubbles with different drug loadings may also be used. In a broader aspect, the invention thus extends to the use of a plurality of microbubbles having different drug payloads. For example, each chemotherapeutic agent may be carried on a separate microbubble. Alternatively, two of the chemotherapeutic agents may be carried on a single microbubble and the remaining chemotherapeutic may be carried on a separate microbubble. It will be understood, that various combinations are possible depending on the choice of chemotherapeutic agents and their means for attachment to the bubble, e.g. whether these are encapsulated or linked to the bubble. Any such combinations of separate microbubble-complexes and their use in any of the methods of treatment herein described also form part of the invention. Where separate microbubbles are used to carry the agents, these will typically be combined to form a single pharmaceutical preparation prior to administration to the patient.

The invention will now be described further with reference to the following non-limiting Examples and the accompanying figures in which:

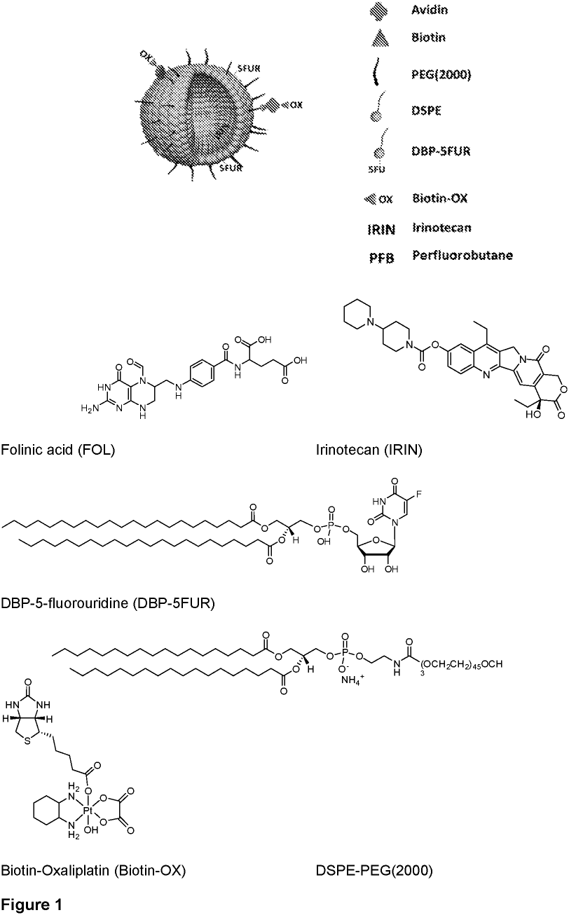

FIG. 1 shows a schematic of FIRINOX-loaded MBs and their constituents.

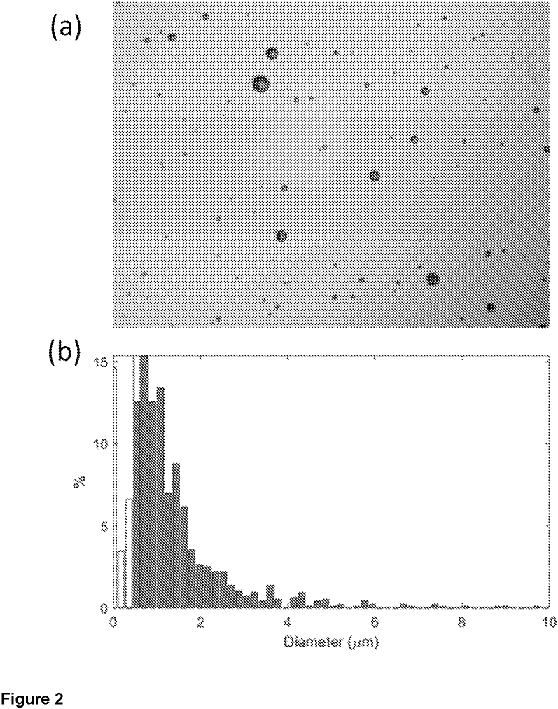

FIG. 2 shows (a) optical microscopy image of FIRINOX MBs; and (b) their size distribution.

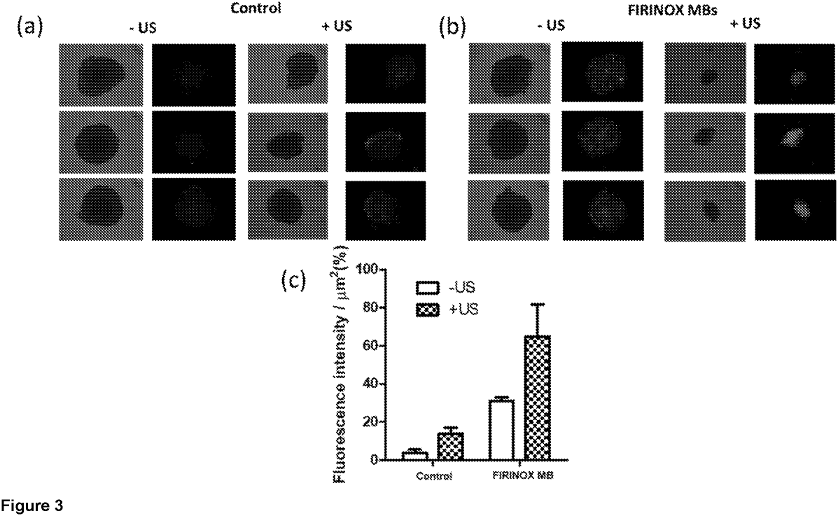

FIG. 3 shows optical (grey panels) and fluorescence (black panels) microscopy images of Panc-01 3D spheroids treated with (+US) or without (-US) ultrasound treatment in the absence (a) or presence (b) of FIRINOX MBs. FIRINOX MBs contained 50 µMIrinotecan, 90 µM 5-fluorouracil and 48 µM Oxaliplatin. Ultrasound conditions: Sonidel SP100 sonoporator with US gel used to mediate contact. Each well was treated with US for 30 secs using a frequency of 1 MHz, an US power density of 3.0 W/cm² and a duty cycle of 50% (pulse frequency = 100 Hz). (c) plot of mean propidium iodide fluorescence intensity per micron of spheroid for each of the groups shown in (a) and (b).

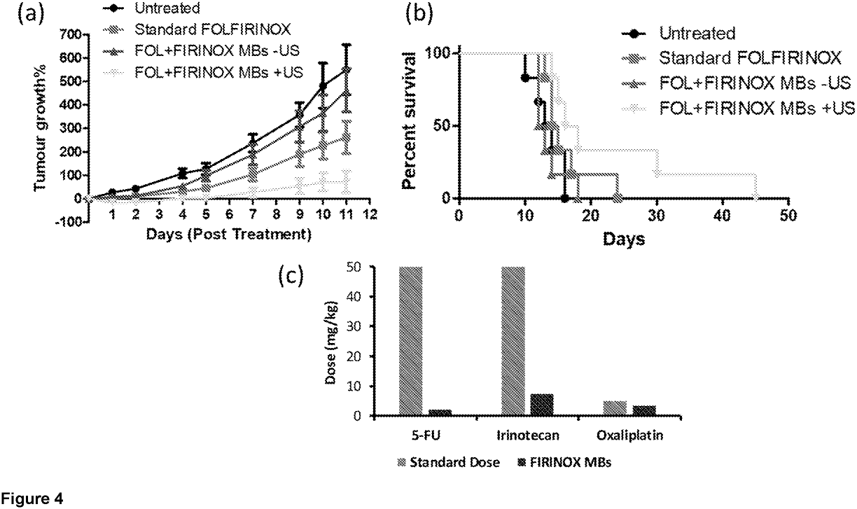

FIG. 4 shows (a) tumour growth delay and (b) Kaplan-Meier survival plot for animals treated in the following manner: (i) Group 1 received no treatment; Group 2 received a standard dose of FOLFIRINOX (folinic acid 100 mg/kg; 5-fluorouracil 50 mg/kg; Irinotecan 50 mg/kg and Oxilaplatin 5 mg/kg) by IV injection; Group 3 received folinic acid (100 mg/kg) and FIRINOX MBs containing 5-fluorouracil (2.1 mg/kg); Irinotecan (7.3 mg/kg) and Oxaliplatin (3.4 mg/kg) by IV injection; Group 4 received the same treatment as Group 3 but with ultrasound directed at the tumour to burst the MBs and release the drug payloads. (c) Graphical representation of the concentrations of the three chemotherapies used in the standard treatment and in the FIRINOX MBs.

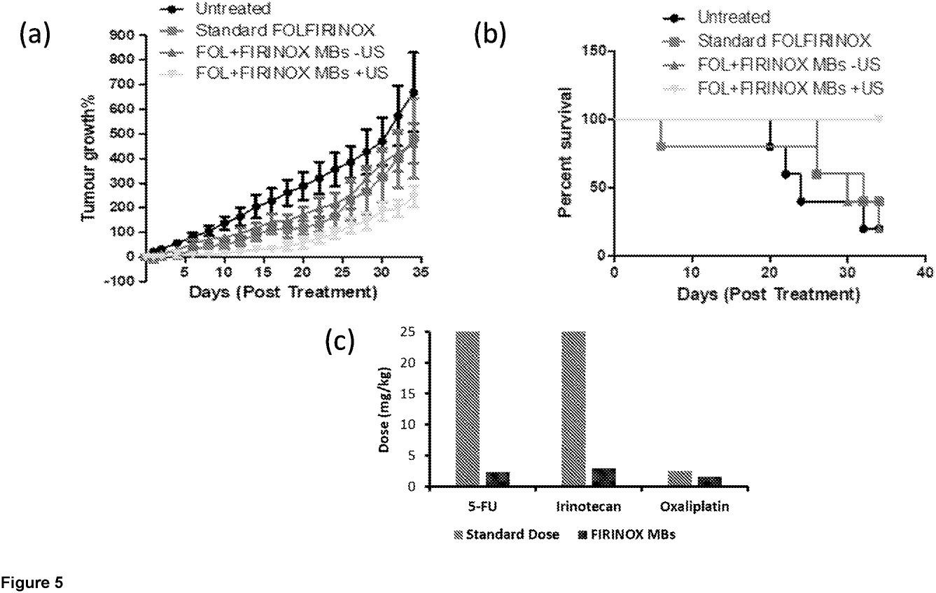

FIG. 5 shows (a) tumour growth delay and (b) Kaplan-Meier survival plot for animals bearing subcutaneous HT-29 colon tumours treated with: (i) Group 1 received no treatment; Group 2 received a standard dose of FOLFRINOX; (ii) Group 3 received folinic acid and FIRINOX MBs; and Group 4 received the same treatment as Group 3 but with ultrasound directed at the tumour to burst the MBs and release the drug payloads. (c) Graphical representation of the concentrations of the three chemotherapies used in the standard treatment and FIRINOX MBs.

### EXAMPLES

### Example 1 - Preparation of FIRINOX-Loaded MBs and Characterisation

A single microbubble (MB) formulation carrying 5-flurouracil, irinotecan and oxaliplatin (FIRINOX) was produced by loading irinotecan (lRIN) hydrophobically into MB shells prepared from a 5-fluorouracil analogue functionalised lipid (F) and other lipids. The resulting FIRIN loaded MBs were attached to an oxaliplatin analogue (OX) using the biotin-avidin interaction to produce the FIRINOX loaded MBs.

### 1.0 Method

All materials were purchased from commercial sources with the exception of DBP-5 FU and Biotin-Ox which were chemically synthesised.

### 1.1 Synthesis of DBP-5FU (“FUR-Lipid”)

A CHCl₃ solution (30 mL) of 1,2-didocosanoyl-sn-glycero-3-phosphocholine (DBPC) (500 mg) was added to a solution of Phospholipase D (PLDP) (10 mg) and 5-fluorouridine (720 mg) in sodium acetate buffer (200 mM pH 5.7 10 mL) containing CaCl₂ (250 mM). The mixture was stirred at 45° C. for 6 hours, then a mixed solution of 2N HCl (5 mL), MeOH (20 mL) and CHCl₃ (20 mL) was added, and the mixture was shaken. The separated organic layer was washed with H₂O (2×10 mL) and then evaporated to dryness. The residue was purified by flash chromatography (silica gel CHCl₃:MeOH 10:1 followed by 6:1) and fractions containing the desired product “FUR-lipid” combined and evaporated to dryness. ¹H NMR (500 MHz, CDCl₃:CD₃OD (2:1) d (ppm) 8.01 (d, 1H, CONHCO), 5.91 (br d, 1H, 1′(CH)), 5.20 (m, 1H, glycerol CH), 3.72-4.20 (m, 9H, 3′(CH), 2′(CH) 4′(CH), 5′(CH₂) glycerol CH₂, glycerol CH₂OPO), 2.27 (m, 4H, 2x COCH₂), 1.57 (m, 4H, 2×CH₂), 1.23 (m, 72H, behenoyl CH₂), 0.83 (t, 6H, 2×CH₃). -ve mode MALDI-MS: Expected for C₅₆H₁₀₂O₁₃N₂P₁F₁ = 1060.71 Found 1059.48

### 1.2 Synthesis of Biotin-OX

(+)-Biotin N-hydroxysuccinimide ester (0.1 g, 0.293 mmol) in anhydrous DMSO (4 mL) was added to a suspension of cis,cis,trans-[Pt(DACH)(Ox)(OH)₂] (0.126 g, 0.305 mmol) in anhydrous DMSO (8 mL). The reaction was stirred at room temperature for 4 days under an argon atmosphere. A small amount of white solid was filtered. The yellow filtrate was concentrated using a DMSO trap to give a sticky yellow oil, to which acetone (40 mL) as added, precipitating a white solid. The suspension was stirred for 1 hr and subsequently the solid was filtered, washed, with acetone, diethyl ether and dried. Yield: 0.122 g (0.186 mmol, 60%). ¹H NMR (400 MHz, DMSO-d₆) δ: 8.63 (s, 1H, NH), 8.19 (s, 1H, NH), 7.86 (s, 1H, NH), 7.18 (s, 1H, NH), 6.38 (d, 2H, 3J = 16 Hz), 4.29 (t, 1H, 3J = 8 Hz), 4.11 (t, 1H, 3J = 8 Hz), 3.06 (dt, 1H, 3J = 8 Hz & 4J = 2 Hz), 2.79 (dd, 1H, 3J = 8 Hz & 4J = 4 Hz), 2.58 (d, 1H, 3J = 4 Hz), 2.54 (s, 2H), 2.16 (t, 2H, 3J = 8 Hz), 1.25 (m, 10H), 1.07 (m, 2H,) ppm.

¹⁹⁵Pt NMR (86 MHz, DMSO-d₆) δ: 1406.7 ppm.

EA calc. % for C₁₈H₃₀ N₄O₈PtS.1.5 H₂O requires C, 31.58; H, 4.86; N, 8.18; S 4.68, found C, 31.60; H, 4.66; N, 7.84; S, 5.00 %.

ESI-MS: m/z ([M+H]+) 658.1 ([M+Na]+) 680.1.

### 1.3 Preparation of FIRINOX MBs

FlRINOX MBs were prepared by first dissolving DBP-5FU (4.0 mg, 3.77 µmol), 1,2-distearoyl-sn-glycero-3-phosphoethanolamine-N-[methoxy(polyethylene glycol) -2000] (DSPE-PEG(2000)) (1.15 mg, 0.41 µmol) and 1,2-distearoyl-sn-glycero-3-phosphoethanolamine-N-[biotinyl(polyethylene glycol)-2000] (ammonium salt) (DSPE-PEG (2000)-biotin) (1.24 mg, 0.41 µmol) in chloroform to achieve a molar ratio of 82:9:9. To this solution was added Irinotecan free base (10 mg) dissolved in chloroform (100 µL). The solvent was removed under vacuum at room temperature yielding a translucent film. The film was then reconstituted in 2 mL of a solution containing PBS, glycerol and proplyene glycol (8:1:1 vol ratio) and heated in a water bath at 80° C. for 30 min. The suspension was sonicated using a Microson ultrasonic cell disrupter at an amplitude of 22% for 1 min and then at an amplitude of 90% in a perfluorobutane (PFB) atmosphere for 30 sec. The MBs were then cooled on ice for 10 min followed by centrifugation at 100 rcf for 3 min and the liquid laying below the surface of the MB cake (infranatant) was removed. The MB cake was then washed a further 2 times with PBS (pH 7.4 ± 0.1) before being mixed for 5 min on ice with an aqueous solution of avidin (10 mg/mL) using an orbital shaker (150 rpm). The MBs were then centrifuged (100 rcf) for 3 min, the infranatant removed and the MB cake washed with PBS solution (2 mL, pH 7.4 ± 0.1) which was again removed following centrifugation. The MB cake was again reconstituted in PBS solution (2 mL, pH 7.4 ± 0.1), mixed for 5 min with an aqueous solution containing Biotin-Ox (1 mL, 5 mg/mL) and centrifuged (100 rcf) for 3 min. Following removal of the infranatant, the MB cake was then washed with PBS (2 mL, pH 7.4 ± 0.1), centrifuged and the MB cake isolated. This washing/centrifugation procedure was repeated twice further with the FIRINOX MB cake reconstituted in 2 mL of PBS. FIG. 1 shows a schematic of the FIRINOX-loaded MBs and their constituents.

### 1.4 Characterisation of FIRINOX-Loaded MBs

The FIRINOX-loaded MBs were characterised by optical microscopy and had a mean particle diameter of 1.14 µm ± 1.17 µm and a concentration of 6.33 × 10⁹ / mL (FIG. 2).

### Example 2 - In Vitro Cytotoxicity of Ultrasound Activated FIRINOX-Loaded Microbubbles in a Panc-01 3D Spheroid Model of Pancreatic Cancer

Cytotoxicity of ultrasound activated FIRINOX MBs prepared in Example 1 was determined in a Panc-01 3D spheroid model of pancreatic cancer.

### 2.1 Method

96 well plates were coated with agarose solution (15 mg/ml in Dulbecco’s Modified Eagle’s Medium (DMEM) - low glucose, 60 µL/well) and air-dried in a laminar-flow hood for 30 min. Panc-01 cells were maintained in Dulbecco’s Modified Eagle’s Medium (DMEM) containing high glucose (4.5 g/L) which were supplemented with 10% (v/v) foetal bovine serum in a humidified 5% CO₂ atmosphere at 37° C. 6×10³ Panc-01 cells were seeded in each well and placed in an incubator (37° C., 5% CO₂) for 96 h to generate the spheroids. The spheroids were then treated with a PBS : medium (50:50 v/v) solution containing the FIRINOX MBs (50 µM IR + 90 µM FUR + 48 µM OX) and selected wells treated with ultrasound delivered using a Sonidel SP100 sonoporator (1 MHz, 30 s, 3 W/cm², duty cycle=50%, and PRF=100 Hz) for 30 secs from underneath the plate using ultrasound gel to mediate contact. Untreated spheroids and spheroids treated with ultrasound only were used for comparative purposes. Following treatment, the spheroids were incubated for a further 48 h when the medium was removed and spheroids then washed 3 times with PBS. The spheroids were then treated with a solution of propidium iodide in PBS (100 µg/ml) and incubated for 30 min after which time the propidium iodide solution was removed and the spheroids washed 3 times with PBS. Micrographic images were recorded using a NIKON Eclipse E400 phase contrast microscope in bright field and fluorescence modes (540 nm band pass excitation and 590 nm long pass emission filters). Image J software was used to quantify propidium iodide fluorescence and it was expressed as a % of P.I. fluorescence intensity/µm², i.e. the propidium iodide fluorescence was normalized according to the area of the spheroid.

### 2.2 Results

The results are shown in FIG. 3 and revealed that spheroids treated with the FIRINOX MBs in combination with ultrasound were significantly smaller while the remaining cells were much brighter (propidium iodide staining indicating cell death) than spheroids treated with FIRINOX MBs alone (i.e. without ultrasound) or spheroids that remained untreated. These results suggest that the physical events that accompany the MB cavitation (bursting) help disperse the three chemotherapies much deeper into the spheroid matrix enhancing their cytotoxicity.

### Example 3 - In Vivo Cytotoxicity of Ultrasound Activated FIRINOX-Loaded Microbubbles in Mice Bearing Subcutaneous KPC Pancreatic Tumours

Cytotoxicity of ultrasound activated FIRINOX MBs prepared in Example 1 was determined using C57 mice bearing ectopic KPC pancreatic tumours that were generated using the T110299 cell line derived from a KPC mouse strain (Duewell et al., 2015, Oncolmmunol. 4:10 e 1029698).

### 3.1 Method

All animals employed in the study were treated humanely and in accordance with the licenced procedures under the UK Animals (Scientific Procedures) Act 1986. KPC cells were maintained in DMEM medium supplemented with 10% foetal calf serum. Cells (5 ×10⁵) were re-suspended in PBS and implanted into the rear dorsum of female C57 mice. Tumour formation occurred approximately 2 weeks after implantation and once tumours became palpable, dimensions were measured using Vernier callipers. Tumour measurements were taken every other day using calipers. Tumour volume was calculated using the equation: tumour volume = (length x width x height)/2. Once tumours reached approximately 100 mm³, animals were separated into the following groups:
Group 1 - no treatment.
Group 2 - a tail vein injection of FOLFIRINOX free drug, i.e. not on a MB. Oxaliplatin at a dose of 5.0 mg/kg was administered first and immediately followed by leucovorin (folinic acid) at a dose of 100 mg/kg, with the addition, after 30 minutes, of irinotecan at a dose of 50 mg/kg, then the treatment was immediately followed by 5-fluorouracil at a dose of 25 mg/kg, administered intravenously).
Group 3 - leucovorin (folinic acid) at a dose of 50 mg/kg administered intravenously followed by FIRINOX MBs ([IRIN]=7.3±1.50 mg/kg, [OX]=3.35±0.37 mg/kg, [FUR]=2.09±0.19 mg/kg) injected intravenously with ultrasound applied to the tumour during and after injection for a total of 3.5 min. Ultrasound was administered using a Sonidel SP100 sonoporator (3.5 W/cm², 1 MHz, 30% duty cycle, and PRF = 100 Hz; PNP = 0.48 MPa; M.I. = 0.48) and ultrasound gel used to mediate contact.
Group 4 - the same treatment as for Group 3 but without ultrasound. Animals were treated on days 0, 3, 6, 8 and both the tumour volume and body weight measurements recorded at the indicated times.

### 3.2 Results

The results are shown in FIG. 4 and reveal a significant improvement in tumour growth delay for animals in Group 4 compared to the other three groups (FIG. 4a). In addition, the animals in Group 4 also survived much longer than those in the other three groups (FIG. 4b).

### Example 4 - In Vivo Cytotoxicity of Ultrasound Activated FIRINOX-Loaded Microbubbles in Mice Bearing Subcutaneous HT-29 Colon Tumours

Cytotoxicity of ultrasound activated FIRINOX MBs prepared in Example 1 was determined in mice bearing subcutaneous HT-29 colon tumours.

### 4.1 Method

All animals employed in this study were treated humanely and in accordance with the licenced procedures under the UK Animals (Scientific Procedures) Act 1986. HT-29 cells were maintained in DMEM medium supplemented with 10% foetal calf serum. Cells (1 x10⁶) were re-suspended in 100 µL of Matrigel® and implanted into the rear dorsum of female Balb/c SCID (C.B-17/IcrHan®Hsd-Prkdcscid) mice. Tumour formation occurred approximately 4 weeks after implantation and once tumours became palpable, dimensions were measured using Vernier callipers. Tumour measurements were taken every other day using calipers. Tumour volume was calculated using the equation: tumour volume = (length x width x height)/2. Once tumours reached approximately 100 mm³, animals were separated into the following groups:
Group 1 - no treatment.
Group 2 - an intraperitoneal injection of FOLFIRINOX free drug treatment (oxaliplatin at a dose of 2.5 mg/kg was administered first and after 2 hours followed by leucovorin (folinic acid) at a dose of 50 mg/kg, immediately followed by 5-fluorouracil at a dose of 25 mg/kg and irinotecan at a dose of 25 mg/kg).
Group 3 - FIRINOX MBs ([IRIN]=2.95±2.04 mg/kg, [OX]=1.60±0.27 mg/kg, [FUR]=2.34±0.20 mg/kg) injection intravenously with ultrasound applied to the tumour during and after injection for a total of 3.5 min followed by an IP injection of leucovorin (folinic acid) at a dose of 50 mg/kg. Ultrasound was administered using a Sonidel SP100 sonoporator (3.5 W/cm², 1 MHz, 30% duty cycle, and PRF = 100 Hz; PNP = 0.48 MPa; M.I. = 0.48) and ultrasound gel used to mediate contact.
Group 4 - the same treatment as for Group 3 but without ultrasound applied to the tumour during treatment.

Animals were treated on days 0, 3, 7, 13, 17 and both the tumour volume and body weight measurements recorded at the indicated times.

### 4.2 Results

The results are shown in FIG. 5 and illustrate an improved tumour growth delay and survival advantage for animals receiving the treatment in accordance with the invention when compared to standard FOLFIRINOX treatment.

1. A microbubble-chemotherapeutic agent complex which comprises a microbubble carrying a combination of chemotherapeutic agents for use in a method of treating cancer in a patient, wherein said combination of chemotherapeutic agents comprises:
(a) a 5-fluoropyrimidine or a derivative thereof;
(b) irinotecan or a derivative thereof; and
(c) a platinum-based chemotherapeutic agent or a derivative thereof;
and wherein said method comprises simultaneous, separate or sequential use of folinic acid or a derivative thereof.

2. A complex for use as claimed in claim 1, wherein said 5-fluoropyrimidine is 5-fluorouracil (5-FU), 5-fluorouridine (5-FUR), capecitabine, carmofur, doxifluridine, tegafur, or a pharmaceutically acceptable salt thereof.

3. A complex for use as claimed in claim 1 or claim 2, wherein said combination of chemotherapeutic agents comprises irinotecan in the form of its free base.

4. A complex for use as claimed in any one of claims 1 to 3, wherein said platinum-based chemotherapeutic agent is cisplatin, oxaliplatin, carboplatin, satraplatin, picoplatin, tetraplatin, platinum-DACH, or a derivative thereof.

5. A complex for use as claimed in claim 4, wherein said platinum-based chemotherapeutic agent is oxaliplatin or Pt(DACH)(Ox)(OH)₂ (wherein DACH = 1,2-diaminocyclohexane, and Ox = oxalate).

6. A complex for use as claimed in any one of the preceding claims, wherein the microbubble has a diameter in the range of from 0.1 to 100 um.

7. A complex for use as claimed in any one of the preceding claims, wherein the microbubble comprises a shell which retains a gas selected from perfluorobutane, perfluoropropane, and oxygen.

8. A complex for use as claimed in any one of the preceding claims, wherein the microbubble has a shell comprising one or more phospholipids, each optionally linked to one or more polymers such as polyethylene glycol (PEG).

9. A complex for use as claimed in any one of the preceding claims, wherein one or more of said chemotherapeutic agents are attached to the microbubble via a non-covalent linkage, e.g. via a biotin-avidin-biotin interaction.

10. A complex for use as claimed in claim 9, wherein said platinum-based chemotherapeutic or derivative thereof is attached to the microbubble via a biotin-avidin-biotin interaction.

11. A complex for use as claimed in any one of the preceding claims, wherein one or more of said chemotherapeutic agents are attached to the microbubble via a covalent linkage.

12. A complex for use as claimed in claim 11, wherein said 5-fluoropyrimidine or derivative thereof (e.g. 5-FUR) and/or said platinum-based chemotherapeutic agent or derivative thereof (e.g. oxaliplatin or Pt(DACH)(Ox)(OH)₂) are attached to the microbubble via a covalent linkage.

13. A complex for use as claimed in claim 11, wherein each of said chemotherapeutic agents is attached to the microbubble via a covalent linkage, preferably wherein all of the following agents are attached to the microbubble via a covalent linkage: 5-FUR, irinotecan, and oxaliplatin or Pt(DACH)(Ox)(OH)₂.

14. A complex for use as claimed in any one of claims 1 to 12, wherein one or more of said chemotherapeutic agents are incorporated into the shell of the microbubble, preferably wherein irinotecan is incorporated into the shell of the microbubble.

15. A complex for use as claimed in any one of the preceding claims, wherein said complex is delivered to affected cells or tissues of said patient and subjected to ultrasound irradiation whereby to rupture the microbubble.

16. A complex for use as claimed in any one of the preceding claims in the treatment of cancer or metastatic cancer, preferably in the treatment of a deep-sited tumour or metastasis derived from said tumour.

17. A complex for use as claimed in claim 16, wherein said cancer is a carcinoma, such as pancreatic adenocarcinoma (PAC) or metastatic pancreatic adenocarcinoma (mPAC).

18. A complex for use as claimed in any one of the preceding claims, wherein one or more of said chemotherapeutic agents are administered to said patient at a sub-therapeutic dosage, for example wherein each of said chemotherapeutic agents is administered at a sub-therapeutic dose.

19. A complex for use as claimed in any one of the preceding claims, wherein said patient has an Eastern Cooperative Oncology Group (ECOG) performance status (PS) which is greater than 1, for example an ECOG PS of 2 or greater.

20. A complex for use as claimed in any one of the preceding claims, wherein said patient suffers from hepatic or renal dysfunction.

21. A complex for use as claimed in any one of the preceding claims as a second-line treatment of cancer.

22. A microbubble-chemotherapeutic agent complex comprising a microbubble carrying a combination of the following chemotherapeutic agents:
(a) a 5-fluoropyrimidine or a derivative thereof;
(b) irinotecan or a derivative thereof; and
(c) a platinum-based chemotherapeutic agent or a derivative thereof.

23. A complex as claimed in claim 22, wherein the microbubble is covalently linked to the 5-fluoropyrimidine or derivative thereof, covalently or non-covalently linked to the platinum-based chemotherapeutic agent or derivative thereof (e.g. linked via a biotin-avidin-biotin interaction), and has a shell in which irinotecan or a derivative thereof is embedded.

24. A method for the preparation of a microbubble-chemotherapeutic agent complex as claimed in claim 22 or claim 23, wherein said method comprises the following steps:
(i) providing a lipid which is capable of forming a microbubble;
(ii) optionally covalently linking one or more chemotherapeutic agents to said lipid whereby to form a functionalised lipid;
(iii) preparing a microbubble from said functionalised lipid, optionally in the presence of one or more chemotherapeutic agents; and
(iv) optionally linking the resulting microbubble to one or more chemotherapeutic agents via a non-covalent linkage, e.g. via a biotin-avidin-biotin interaction.

25. A pharmaceutical composition comprising a microbubble-complex as claimed in claim 22 or claim 23, and folinic acid or a derivative thereof, together with one or more pharmaceutically acceptable carriers or excipients.
